(19) United States
(12) Patent Application Publication
(10) Pub. No.: US 2024/0343779 A1
(43) Pub. Date: Oct. 17, 2024

(54) PHARMACOLOGICAL AND/OR GENETIC ACTIVATOR FOR USE IN PRESERVING AND REGENERATING MUSCLE STRUCTURE AND FUNCTION BY BLOCKING SENESCENCE

(71) Applicants: ASSOCIATION FRANCAISE CONTRE LES MYOPATHIES, Paris Cedex 13 (FR); UNIVERSITÉ PARIS-EST CRÉTEIL (UPEC), Créteil (FR); INSTITUT NATIONAL DE LA SANTE ET DE LA RECHERCHE MEDICALE (INSERM), Paris (FR)

(72) Inventors: Valentina TAGLIETTI, Paris (FR); Frédéric RELAIX, PERIGNY SUR YERRES (FR)

(73) Assignee: INSTITUT NATIONAL DE LA SANTE ET DE LA RECHERCHE MEDICALE (INSERM), Paris (FR)

(21) Appl. No.: 18/294,626

(22) PCT Filed: Aug. 4, 2022
(86) PCT No.: PCT/EP2022/071914

§ 371 Date: Feb. 2, 2024

(30) Foreign Application Priority Data
Aug. 4, 2021 (FR) FR2108489

(51) Int. Cl. C07K 14/72; A61K 31/352; A61K 48/00; C12N 15/86; C12N 15/88

(52) U.S. Cl. CPC C07K 14/723; A61K 31/352; C12N 15/86; C12N 15/88; A61K 48/00; C12N 2750/14143

(57) ABSTRACT

An activator for preserving and regenerating muscle structure and function activates the thyroid-stimulating hormone receptor (TSHR) signaling pathway in muscle stem cells (satellite cells), thereby blocking senescence. A medical product includes a plurality of activators, such as a genetic activator, and an adenoviral vector comprising a thyroid-stimulating hormone receptor (TSHR) protein sequence of SEQ ID NO: 1, or a TSHR nucleotide sequence of SEQ ID NO: 2.

### TECHNICAL FIELD

The invention relates to a novel pharmacological and/or genetic activator for use in preserving and regenerating muscle structure and function by blocking senescence, in particular for preventing and/or treating myopathies, preferably Duchenne muscular dystrophy (DMD), as well as sarcopenia. The invention also relates to a novel adenoviral vector for use as a medicinal product.

### PRIOR ART

Muscular dystrophy is a progressive hereditary neuromuscular disease, resulting from the mutation of one or more genes responsible for muscle function and structure.

Duchenne muscular dystrophy is the most widespread myopathy in children. It is due to abnormalities of the protein dystrophin that is essential to the muscles. It is characterized by a decline in muscle regeneration contributing to the loss of muscle mass.

In patients suffering from Duchenne myopathy, in the absence of dystrophin, the fibers that constitute the skeletal muscles, the smooth muscles and the cardiac muscle become damaged with each contraction and are eventually destroyed.

Duchenne muscular dystrophy is due to a recessive genetic abnormality in the DMD gene, on chromosome X, leading to the absence of dystrophin.

Dystrophin is a large protein which ensures the link between the contractile cytoskeleton and the extracellular matrix, via a protein complex called the dystrophin membrane complex, and plays a major role in maintaining the integrity of muscle fibers during contractions.

Dystrophin is a protein made up of 3,685 amino acids with a molecular weight of 427 kDa. It is encoded by the DMD gene which allows the synthesis of 7 main isoforms of different sizes, by virtue of the presence of 7 tissue-specific promoters.

Three of these promoters (called M, B and P promoters, indicating an expression in Muscle, Brain and Purkinje cerebellar neuron tissues) are located upstream of the first exon and allow the synthesis of functionally equivalent full-length dystrophins.

The other four promoters are intragenic and allow the synthesis of smaller isoforms.

Dystrophin is part of the family of “spectrin” type filamentous proteins that are present in skeletal and cardiac muscle tissues, the brain, the retina, glial cells and Purkinje cells and in a very small amount in lymphocytes.

Dystrophin consists of four large domains which interact with several partners of the muscle cell and play an essential role during the muscular contraction-relaxation cycle:
the N-terminal domain or actin binding domain (ABD1), comprising the first 246 amino acids;
the central domain (or rod domain) ranging from amino acid 247 to 3,045. It consists of 24 spectrin-like repeats (R1 to R24) and of 4 hinges rich in proline (H1 to H4) dividing the central rod domain into 3 subdomains;
the cysteine-rich domain (cysteine rich repeats or CR) ranging from amino acid 3,080 to 3,360; and
the C-terminal domain formed of the 325 last residues.

Many clinical trials are in progress for the treatment of Duchenne muscular dystrophy using gene or pharmacological therapy.

The aim of gene therapy is to produce functional dystrophin in the muscle.

A first clinical trial began recently in France. It is evaluating a gene therapy product that combines a shortened version of the DMD gene with an AAV adeno-associated viral vector.

Pharmacological therapies act on certain manifestations of the disease to slow down its progression. Several medicinal products are being studied to improve and/or protect muscle function and/or cardiac function, combat inflammation and the effects of the absence of dystrophin on muscle cells: tamoxifen, nebivolol, givinostat, rimeporide, etc.

Patent document WO2018155913 relates to a composition comprising chemical compounds selected from the inhibitors of the pan-histone deacetylase, ALK5 inhibitors (kinase 5 inhibitor similar to the activin A receptor) and cAMP signaling activators making it possible to induce differentiation of somatic cells, mesenchymal stem cells or adult stem cells (with the exception of muscle stem cells) into skeletal muscle cells for the treatment of skeletal muscle diseases. It implements a method in which cells are cultured in a culture medium containing the composition inducing differentiation into skeletal muscle cells. The skeletal muscle cells thus obtained have no risk of oncogenesis after implantation due to differentiation from somatic cells, and have the advantage of being genetically stable due to the use of a mixture of compounds of low molecular weight alone, without the introduction of genetics.

### Technical Problem

Considering the foregoing, a problem that the present invention proposes to solve consists in developing a new therapy for preserving and regenerating muscle structure and function and for treating, in particular, Duchenne muscular dystrophy.

To date, there is no known therapy aimed at targeting cellular senescence of muscle stem cells to treat Duchenne muscular dystrophy. Muscular stem cell senescence is one of the effects caused by Duchenne muscular dystrophy (Sugihara, Hidetoshi et al. “Cellular senescence-mediated exacerbation of Duchenne muscular dystrophy.” Scientific Reports vol. 10,1 16385. Oct. 12, 2020, do:10.1038/s41598-020-73315-6). This contributes to the strong reduction in muscle regeneration and thus causes the worsening of the pathology.

### Technical Solution

The first subject matter aimed at solving this problem is a pharmacological and/or genetic activator of the thyroid-stimulating receptor hormone (TSHR) signaling pathway in muscle stem cells (satellite cells) for use in preserving and regenerating muscle structure and function by blocking senescence.

Surprisingly, targeting TSHR signaling in a pharmacological and/or genetic manner makes it possible to preserve the capacities of proliferation and differentiation of muscle stem cells by blocking senescence. The activation of TSHR signaling in muscle stem cells increases the regeneration properties of the muscles affected in particular by Duchenne muscular dystrophy and preserves muscle structure and function, by preventing rapid muscle wasting which occurs with the progression of the pathology.

The invention also relates to a combination of a pharmacological activator and a genetic activator for their uses according to the invention.

The invention also relates to an adenoviral vector, characterized in that it comprises a thyroid-stimulating receptor (TSHR) protein sequence of SEQ ID NO: 1 or a functional variant having a protein sequence at least 60% identical to the sequence SEQ ID NO: 1, or a TSHR nucleotide sequence of SEQ ID NO: 2 or a functional variant having a nucleotide sequence at least 60% identical to the sequence SEQ ID NO: 2 for use thereof as a medicinal product.

### Advantages Provided

The applicant has in particular been able to develop a pharmacological and/or genetic activator of the thyroid-stimulating hormone receptor (TSHR) signaling pathway in muscle stem cells making it possible to increase muscle regeneration by preventing the loss of muscle mass and substitution by adipose and fibrous tissues. This approach will make it possible to maintain the functionality of the muscles and to slow down the progression of Duchenne muscular dystrophy.

### BRIEF DESCRIPTION OF THE DRAWINGS

The invention and its advantages will be better understood on reading the following description and non-limiting embodiments, shown with reference to the appended drawings, in which:

FIG. 1 shows the generation of the R-DMDdel52 rats as detailed in example 1 in which:
FIG. 1A is an observation of dystrophin and β-dystroglycan (gray) by immunofluorescence at the tibialis anterior of the WT rats and the R-DMDdel52 rats at ages 3 weeks and 12 months. (Scale Bar=to 20 μm).
FIG. 1B is a Western Blot analysis of the expression rate of the dystrophin at the tibialis anterior of one WT rat and two R-DMDdel52 rats (DMD #1, DMD #2) at age 16 weeks. β-Tubulin is used as a housekeeping gene.
FIG. 1C represents a survival curve of the WT rats and of the DMD rats by the Kaplan Meier estimator.
FIG. 1D details the staining of the tibialis anterior muscle tissue using hematoxylin and eosin (top panel) and Sirius red (bottom panel), originating from 12-month-old WT and R-DMDdel52 rats. (Scale bar=20 μm).
FIG. 1E represents the quantification of fibrous tissue deposition by the percentage of Sirius red-stained surface at the tibialis anterior in 12-month-old WT and R-DMDdel52 rats. Average±standard deviation (n=3), Mann-Whitney test.
FIG. 1F corresponds to the percentage of central nucleus myofibrils at the tibialis anterior of the 12-month-old WT and R-DMDdel52 rats. Average±standard deviation (n=3), Mann-Whitney test.
FIG. 1G corresponds to the percentage of distribution of muscle fiber sizes of the cross-section at the tibialis anterior in 12-month-old WT and R-DMDdel52 rats. Average±standard deviation (n=3), ANOVA test. The p values are calculated by the Sidak post-hoc test.
FIG. 1H details the staining of the extra-ocular muscles, using hematoxylin and eosin (top panel, in white) and Sirius red (bottom panel, in black), originating from 12-month-old WT and R-DMDdel52 rats. (Scale bar=20 μm).
FIG. 1I represents the quantification of fibrous tissue deposit by the percentage of Sirius red-tinted surface at the extra-ocular muscles in WT rats and R-DDdel52 of the age of 12 months. Average±standard deviation (n=3), Mann-Whitney test.
FIG. 1J corresponds to the percentage of central-nucleus myofibrils at the extra-ocular muscles of the WT rats and R-DMDdel52 at 12 months old. Average±standard deviation (n=3), Mann-Whitney test.
FIG. 1K corresponds to the percentage of distribution of the cross-section at the extra-ocular muscles in 12-month-old WT and R-DMDdel52 rats. Average±standard deviation (n=3), ANOVA test. The p values are calculated by the Sidak post-hoc test.

FIG. 2 shows the muscular continuous regeneration of the progressive extra-ocular muscles in the DMD model of the rat as detailed in example 2, in which:
FIG. 2A is an observation of the light chain of embryonic myosin (eMHC-embryonic myosin heavy chain, gray cluster in the bottom left of the panel) and of the laminin (in the form of a network) in the tibialis anterior of the WT and R-DMDdel52 rats aged 3 weeks, 6 months and 12 months, by immunofluorescence, with the nucleus labeled using Hoechst blue dye. (Scale bar=20 μm).
FIG. 2B corresponds to the quantifications of the number of eMHC per mm2 in the tibialis anterior of the WT and R-DMDdel52 rats aged 3 weeks, 6 months and 12 months. Average±standard deviation (n=3), Mann-Whitney test.
FIG. 2C is an observation of the light chain of eMHC (gray cluster) and the laminin (in the form of a condensed network) at the extra-ocular muscles of the WT and R-DMDdel52 rats aged 3 weeks, 6 months and 12 months, by immunofluorescence, with the nucleus labeled using Hoechst blue dye. (Scale bar=20 μm).
FIG. 2D corresponds to the quantifications of the number of eMHC per mm2 at the extra-ocular muscles of the WT and R-DMDdel52 rats aged 3 weeks, 6 months and 12 months. Average±standard deviation (n=3), Mann-Whitney test.
FIG. 2E corresponds to representative co-immunofluorescence staining at the tibialis anterior, using Ki67 antibodies (white triangle), Pax7 (0 box), laminin (in the form of a network) and Hoechst (the circles in the cells). The yellow boxes indicate a co-expression of Pax7 and Ki67 and the gray rectangles indicate Pax7+/Ki67− of muscle stem cells. (Scale bar=10 μm).
FIG. 2F represents the quantification of E showing the number of muscle cells (PAX7+) per fiber at the tibialis anterior of WT and R-DMDdel52 rats aged 3 weeks, 6 months and 12 months. Average±standard deviation (n=3), Mann-Whitney test.
FIG. 2G represents the quantification of the percentage of quiescent MSC (muscle stem cells) (Pax7+/Ki67−) and proliferative MSC (Pax7+/Ki67+) on the isolated TA of WT and R-DMDdel52 rats aged 3 weeks, 6 months and 12 months. Average±standard deviation (n=3), Mann-Whitney test.

FIG. 3 shows the myogenic potential of the MSCs of the R-DMDdel52 rats of the TA in vitro, in which:
FIGS. 3A and 3B corresponding to a representative co-immunofluorescence staining of Ki67 (round), MyoD (square) and nuclei (Hoechst, star) of myoblasts isolated from the tibialis anterior (A) and extra-ocular muscles (B) of 3-week-old WT and R-DMDdel52 rats. The arrows indicate the proliferative myoblasts co-labeled with Ki67 and MyoD. Scale bar 20 μm.
FIG. 3C corresponds to the quantifications of the myoblasts MyoD+/Ki67+ and MyoD+/Ki67− from the tibialis anterior and the extra-ocular muscles of 3-week-old WT and R-DMDdel52 rats. Average±standard deviation (n=3), Mann-Whitney test.
FIGS. 3D and 3E represent the immunofluorescence labeling of the sarcomeric myosins (MF20 in the form of filaments) and the nuclei (Hoechst), of the myotubes differentiated in the tibialis anterior (D) and of the extra-ocular muscles (E) of 3-week-old WT and R-DMDdel52 rats. Scale bar 20 μm.
FIG. 3F is an evaluation of the field fusion index on differentiated myotubes of the EOM (extra-ocular muscles) at the age of 3 weeks. Average±standard deviation (n=3), Mann-Whitney test.
FIG. 3G represents the number of nuclei per myotube per field (mm2) for the WT and R-DMDdel52 myotubes isolated from TA and EOM at the age of 3 weeks. Average±standard deviation (n=3), Mann-Whitney test.
FIGS. 3H and 3I corresponding to co-immunofluorescence staining of Ki67, MyoD and nuclei (Hoechst) of myoblasts isolated from TA (H) and EOM (I) of 6-month-old WT and R-DMDdel52 rats. The arrows indicate the proliferative myoblasts co-labeled with Ki67 and MyoD, while the stars highlight the MyoD+/Ki67− myoblasts. Scale bar 20 μm.
FIG. 3J represents the quantifications of the MyoD+/Ki67+ and MyoD+/Ki67− myoblasts from the TA and the EOM of 6-month-old WT and R-DMDdel52 rats. Average±standard deviation (n=3), Mann-Whitney test.
FIGS. 3K and 3L corresponding to an immunofluorescence staining of the sarcomeric myosins (MF20, green) and of the nuclei (Hoechst, blue) of the differentiated myotubes of the TA (K) and of the EOM (L) of 6-month-old WT and R-DMDdel52 rats. Scale bar 20 μm.
FIG. 3M is an evaluation of the field fusion index on the differentiated myotubes of the TA and the EOM of 6-month-old rats. Average±standard deviation (n=3), Mann-Whitney test.
FIG. 3N represents the number of nuclei per myotube per field (mm2) for the WT and R-DMDdel52 myotubes isolated from the TA and EOM of the 6-month-old rats. Average±standard deviation (n=3), Mann-Whitney test.
FIG. 3O corresponds to a co-immunofluorescence staining of EdU, Pax7/MyoD and nuclei (Hoechst) of FACS-sorted myoblasts from isolated limb muscles of 6-month-old WT rats and R-DMDdel52 rats. The arrows indicate the proliferative myoblasts co-labeled with Ki67 and MyoD, while the stars highlight the negative MyoD-positive myoblasts for ki67. Scale bar 20 μm.
FIG. 3P corresponds to the percentage of cell type (Pax7+/MyoD+ and Pax7+/MyoD−) to evaluate the purity of the cells after the FACS sorting.
3Q) corresponds to the quantification of the proliferative myoblasts (EdU+) (Pax7:MyoD+) from cells sorted by FACS and isolated from the limb muscles of 6-month-old WT and R-DMDdel52 rats. Average±standard deviation (n=3), Mann-Whitney test.
FIG. 3R is an immunofluorescence staining of sarcomeric myosins (MF20, in the form of a broad band (WT) and a thick line (DMD)) and nuclei (Hoechst, round) on myotubes differentiated from FACS-sorted myoblasts of 6-month-old WT and R-DMDdel52 rats. Scale bar 50 μm.
FIG. 3S corresponds to the quantification of the field fusion index for the experiment carried out in (R).

FIG. 4 shows the expression of senescence markers at the satellite cells of the Tibialis anterior (TA) R-DMDdel52 muscle as detailed in example 4, in which:
FIG. A4 corresponds to a graph plotted by the t-SNE algorithm on the grouping of populations of cells residing in the muscles, revealed by scRNA-seq analysis on the TA of 12-month-old WT and R-DMDdel52 rats.
FIG. 4B corresponds to a t-SNE graph divided between WT and R-DMDdel52.
FIG. 4C corresponds to a t-SNE graph showing the expression of Cdkn1a and Cdkn2a at the muscle stem cells (MSC) of the WT and R-DMDdel52 samples.
FIG. 4D represents violin plots showing the distribution of gene expression of Pax7 and Myf5 as specific genes of MSC and of Cdkn1a and Cdkn2a as senescence markers.
FIGS. 4E-G corresponding to staining of the TA by co-immunofluorescence, using antibodies against Pax7, Cdkn2a (also called p16; star, E) or Cdkn1a (also called p21; star, F) or YH2AX (star, G), laminin (in the form of a network) and Hoechst for the nuclei. The triangles highlight the PAX7+ MSCs. Scale bar 10 μm.
FIG. 4H corresponds to the quantification of E showing the percentage of MSC (Pax7+) co-expressing Cdkn2a at 3 weeks, 6 months and 12 months at the TA of the WT and R-DMDdel52 rats. Average±standard deviation (n=3), Mann-Whitney test.
FIG. 4I corresponds to the quantification of F showing the percentage of MSC (Pax7+) co-expressing Cdkn1a at 3 weeks, 6 months and 12 months at the TA in the WT and R-DMDdel52 rats. Average±standard deviation (n=3), Mann-Whitney test.
FIG. 4J corresponds to the quantification of G showing the percentage of MSC (Pax7+) co-expressing YH2AX at age 3 weeks, 6 months and 12 months at the TA of the WT and R-DMDdel52 rats. Average±standard deviation (n=3), Mann-Whitney test.

FIG. 5 shows the expression of senescence markers at MSCs of human patients suffering from DMD as detailed in example 5, in which:
FIG. 5A represents an immunofluorescence for embryonic myosin heavy chain (eMHC, large gray filled circle) and laminin (in the form of a network) on control human biopsies and DMD at 1-2, 3-4, 5-6, 7-8 years of age. Scale bar 20 μm.
FIG. 5B corresponds to the quantification of the number of eMHC+ myofibers per mm² relative to A. Average±standard deviation (Control n=12; DMD n=20), Mann-Whitney test.
FIG. 5C is a co-immunofluorescence staining of DMD and control muscle biopsies, using antibodies against Pax7, Cdkn2a (red) and Hoechst. The yellow triangles highlight the PAX7+ MSCs. Scale bar 10 μm.
FIG. 5D represents the quantification of C showing the percentage of Pax7 MSC co-expressing Cdkn2a in DMD and control muscle biopsies. Average±standard deviation (Control n=12; DMD n=20), Mann-Whitney test.
FIG. 5E represents a Pearson correlation test showing the correlation between the DMD MSCs expressing Cdkn2a and the age (Control n=12; DMD n=20).
FIG. 5F is a representative co-immunofluorescence staining of DMD and control muscle biopsies, using antibodies against Pax7, YH2AX (star) and Hoechst. The triangles highlight the PAX7+ MSCs. Scale bar 10 μm.
FIG. 5G represents the quantification of F showing the percentage of MSC (Pax7+) co-expressing YH2AX in DMD and control muscle biopsies. Average±standard deviation (Control n=12; DMD n=15), Mann-Whitney test.
FIG. 5H represents a Pearson correlation test between the DMD MSCs expressing YH2AX and age (Control n=12; DMD n=15).

FIG. 6 shows the expression of a specific signature of the MSCs at the EOM distinct from that at the TA level as detailed in example 6, in which:
FIG. 6A is a graph plotted by the t-SNE algorithm of muscle-resident cell population clusters, revealed by scRNA-seq at the TA (left panels) and EOM (right panels) of 12-month-old WT and R-DMDdel52 rats, showing the expression of Pax7, Pitx2, Cdkn1a, Cdkn2a and Tshr. The squares indicate the MSC group.
FIGS. 6B and 6C represent violin plots showing the distribution of gene expression of Pax7, Cdkn1a, Cdkn2a in B and Myf5, Pitx2 and Tshr in C from the scRNAseq analysis carried out at the TA and the EOM of 12-month-old WT and R-DMDdel52 rats.
FIG. 6D is a qPCR analysis of myoblasts derived from EOMs and from the limbs as expression fold relative to myoblasts derived from EOMs for Cdkn1a, Cdkn2a and Tshr. Average±standard deviation (n=3), Mann-Whitney test.
FIGS. 6E-6G correspond to a co-immunofluorescence staining of EOMs, using antibodies against Pax7, Cdkn2a (E) or Cdkn1a (F) or YH2AX (G), laminin (in the form of a network) and Hoechst. The triangles highlight the MSCs (PAX7+). Scale bar 10 μm.
FIG. 6H corresponds to the quantification of E showing the percentage of MSC (Pax7+) co-expressing Cdkn2a at the TA of WT and R-DMDdel52 rats aged 3 weeks, 6 months and 12 months. Average±standard deviation (n=3), Mann-Whitney test.
FIG. 6I corresponds to the quantification of F showing the percentage of MSC (Pax7+) co-expressing Cdkn1a in the TA of WT and R-DMDdel52 rats aged 3 weeks, 6 months and 12 months. Average±standard deviation (n=3), Mann-Whitney test.
FIG. 6J corresponds to the quantification of G showing the percentage of MSC (Pax7+) co-expressing YH2AX in the TA of WT and R-DMDdel52 rats aged 3 weeks, 6 months and 12 months. Average±standard deviation (n=3), Mann-Whitney test.

FIG. 7 shows the regulation of senescence at myoblasts isolated from EOMs in the R-DMDdel2 rats, in which:
FIG. 7A corresponds to an immunofluorescence for TSHR (stars) and Pax7/MyoD on myoblasts of 3-month-old rats isolated from the EOMs (on the left) and the muscles of the limbs (on the right). The nuclei were stained with Hoechst. Scale bar 20 μm.
FIG. 7B is a co-immunofluorescence staining of the EdU (middle panels), Pax7/MyoD (left-hand panels) and nuclei (Hoechst, right-hand panels) of the EOM-derived myoblasts treated with vehicle (DMSO) or ML224. Scale bar 20 μm.
FIG. 7C corresponds to the quantifications of the proliferative myoblasts (EdU+) (Pax7/MyoD+) from EOM-derived myoblasts treated with a vehicle (DMSO) or the inhibitor of TSHR ML224. Average±standard deviation (n=3), Mann-Whitney test.
FIG. 7D is an immunofluorescence staining of sarcomeric myosins (MF20, in the form of a strip) and nuclei (Hoechst) of differentiated myotubes from EOM cells treated with a vehicle (DMSO) or ML224. Scale bar 20 μm.
FIG. 7E is an evaluation of the field fusion index on the differentiated EOM myotubes, treated with the vehicle (DMSO) or ML224. Average±standard deviation (n=4), Mann-Whitney test.
FIG. 7F corresponds to a qPCR analysis on EOM-derived myoblasts treated with a vehicle or ML224, evaluating the genetic expression of Cdkn1a and Cdkn2a. Average±standard deviation (n=4-5), Mann-Whitney test.
FIG. 7G represents a representative co-immunofluorescence staining of Pax7/MyoD (left-hand panels), EdU (middle panels) and nuclei (Hoechst, right-hand panels) on myoblasts isolated from WT and R-DMDdel52 limb muscles treated with a vehicle (DMSO) or with the activator of adenylate cyclase, forskolin. Scale bar 20 μm.
FIG. 7H corresponds to the quantifications of the myoblasts Pax7:MyoD+/EdU+ and Pax7:MyoD+/EdU− originating from WT and R-DMDdel52 myoblasts isolated from the limb muscles after treatment with forskolin or with the vehicle (DMSO). Average±standard deviation (n=3), Mann-Whitney test.
FIGS. 7I and 7J correspond to the qPCR analysis on limb-derived myoblasts treated with a vehicle or with forskolin from WT and R-DMDdel52 rats, evaluating the genetic expression of Cdkn1a (I) and Cdkn2a (J). Average±standard deviation (n=4), Mann-Whitney test.
FIG. 7K represents an immunofluorescence staining of the sarcomeric myosins (MF20, in the form of bands) and nuclei (Hoechst, circles) of differentiated myotubes originating from WT and R-DMDdel52 limb cells treated with a vehicle (DMSO) or with forskolin. Scale bar 20 μm.
FIG. 7L is an evaluation of the field fusion index on differentiated myotubes from the WT and R-DMDdel52 limb muscles, treated with the vehicle (DMSO) or forskolin. Average±standard deviation (n=4), Mann-Whitney test.

FIG. 8A corresponds to the Scheme of the TSHR Tomato lentivirus.

FIG. 8B shows the expression of Tomato in transduced WT and DMD myoblasts with a control lentivirus (Tomato) or with the TSHR Tomato lentivirus.

FIG. 8C represents a qPCR analysis of the transcription levels of the TSHR gene.

FIG. 8D corresponds to the percentage of MyoD-positive myoblasts expressing the p16 senescence marker.

### DESCRIPTION OF EMBODIMENTS

The invention relates to an activator of the thyroid-stimulating hormone receptor (TSHR) signaling pathway in muscle stem cells (satellite cells) for use thereof in preserving and regenerating muscle structure and function by blocking senescence, preferably in humans and animals, even more preferably in humans.

Preferably, the invention relates to a pharmacological and/or genetic activator of the thyroid-stimulating hormone receptor (TSHR) signaling pathway in muscle stem cells (satellite cells) for use in preserving and regenerating muscle structure and function by blocking senescence, preferably in humans and animals, even more preferably in humans.

Surprisingly, the applicant was thus able to identify novel pharmacological and/or genetic activators of the thyroid-stimulating hormone receptor (TSHR) signaling pathway, taken alone or in combination, for their use specifically on muscle stem cells (satellite cells) to block their senescence.

TSHR belongs to the family of glycoprotein receptors of rhodopsin which, when activated, stimulates the adenylate cyclase, activating the cyclic adenosine monophosphate (cAMP)/protein kinase A (PKA) pathway (Morshed et al., “Characterization of thyrotropin receptor anti-induced signal cascades.” Endocrinology vol. 150,1 (2009): 519-29; Rowe et al., “Tezacaftor-Ivacaftor in Residual-Function Heterozygotes with Cystic Fibrosis.” The New England journal of medicine vol. 377,21 (2017): 2024-2035).

Muscular stem cell senescence is one of the effects caused by Duchenne muscular dystrophy (DMD). This contributes to the strong reduction in muscle regeneration and thus causes the worsening of the pathology.

Preferably, preserving and regenerating muscle structure and function by blocking the senescence of muscle stem cells (satellite cells) makes it possible to prevent and/or treat myopathies, more preferably DMD.

According to another embodiment, the pharmacological and/or genetic activators of the TSHR signaling pathway in muscle stem cells (satellite cells) used according to the invention make it possible to prevent and/or treat sarcopenia.

Sarcopenia is understood to mean a geriatric syndrome that is initially characterized by a reduction in muscle capacity due to age and which, as it worsens, will cause a deterioration in muscular strength and physical performance. Sarcopenia observed in an elderly person is attributable to the aging process but can be accelerated by pathological and behavioral factors such as malnutrition and a sedentary lifestyle. From the age of 30, muscular degeneration occurs at a rate of 3 to 8% per decade. Muscle tissue is replaced by fat mass and this loss accelerates from the age of 50. Indeed, muscle mass declines approximately 1 to 2% per year past the age of 50 years, while strength declines on average by 1.5% per year between 50 and 60 years of age (−15%), then at a rate of 3% per year, i.e. a loss of 30% per decade after the age of 60.

According to a preferred embodiment of the invention, the activator of the TSHR signaling pathway in muscle stem cells (satellite cells) used according to the invention is an activator selected from a TSHR receptor agonist or an adenylate cyclase activator, taken alone or in combination.

The TSHR receptor agonist used according to the invention is understood to mean a compound capable of directly activating the TSHR receptor by binding to the TSHR receptor or indirectly via other compounds.

As an example of a TSHR receptor agonist, mention may be made of recombinant human TSH (rhTSH) Thyrogen®, an antibody stimulating TSHR (such as M22), NCGC00161870 known as C2 and consisting of the E1 and E2 enantiomers, NCGC00379308, and MSq1.

The pharmacological activator used according to the invention is preferably an adenylate cyclase activator selected from NKH 477 (6-[3-(dimethylamino)propionyl]forskolin), cw008, DMAPD (6-[3-(dimethylamino)propionyl]-14,15-dihydroforskolin), PACAP-27, PACAP-38, glucagon, isoproterenol, octopamine, PACAP 27 amine, prostaglandin D2/E1/E2/I2, vasopressin, forskolin and its analogs such as 6-acetyl-7-deacetyl-forskolin, 7-deacetyl-7-O-4-hemisuccinyl-forskolin, 7-deacetyl-7-(O—N-methylpiperazino)-γ-butylryl-dihydrochloride-forskolin, 7-deacetyl-forskolin (D3533), 6-[3-(dimethylamino)propionyl]forskolin, 6-[N-(2-isothiocyanatoethyl)aminocarbonyl]forskolin, 5,6-dehydroxy-7-deacetyl-7-nicotinoylforskolin, 6-acetyl-7-deacetyl-forskolin, 7-deacetyl-7-O-hemisuccinyl-forskolin, 1,9-dideoxy-forskolin (D3658), preferably forskolin.

According to another preferred embodiment of the invention, the activator of the TSHR signaling pathway in muscle stem cells (satellite cells) used according to the invention is a genetic activator.

Preferably, the genetic activator used according to the invention consists of a vector selected from a liposome comprising a thyroid-stimulating hormone receptor (TSHR) protein sequence of SEQ ID NO: 1 or a functional variant having a protein sequence at least 60% identical to the sequence SEQ ID NO: 1, an adenoviral vector comprising the protein sequence of SEQ ID NO: 1 or a functional variant having a protein sequence at least 60% identical to the sequence SEQ ID NO: 1, or a TSHR nucleotide sequence of SEQ ID NO: 2 or a functional variant having a nucleotide sequence at least 60% identical to the sequence SEQ ID NO: 2, and an adenoviral vector comprising a CRISPR-Cas9 sequence fused with a transactivator domain preferably selected from VP16, VP64, p65, NCO1A1, FOXO1A to activate the expression of the TSHR.

Nucleotide is understood to mean any polyribonucleotide or polydeoxyribonucleotide, which may be an unmodified RNA or DNA or a modified RNA or DNA. The polynucleotides comprise, without limitation, single-stranded and double-stranded DNA, DNA that is a mixture of single-stranded and double-stranded regions or single-stranded and triple-stranded regions, single-stranded and double-stranded RNA, and RNA which is a mixture of single-stranded and double-stranded regions, hybrid molecules comprising DNA and RNA which can be single-stranded or, more typically, double-stranded, or triple-stranded regions, or a mixture of single-stranded and double-stranded regions. As used herein, the term “polynucleotide(s)” also comprises the DNA or RNA described above that contain one or more modified bases. Thus, the DNA or RNA whose backbones are modified for reasons of stability or for other reasons are “polynucleotides” in the sense that this term is understood here. In addition, the DNA or RNA comprising unusual bases, such as inosine, or modified bases, such as tritylated bases, to mention only two examples, are polynucleotides in the sense that this term is used here. It will be understood that a large variety of modifications were made to DNA and RNA, which serve for numerous useful purposes known to persons skilled in this field. The term “polynucleotide(s)” as used herein encompasses such chemically, enzymatically or metabolically modified polynucleotide forms as well as chemical forms of DNA and RNA characteristic of viruses and cells, including, for example, single and complex cells. The term “polynucleotide(s)” also encompasses short polynucleotides, often called oligonucleotides.

The term protein sequence refers to any peptide or protein comprising two or more amino acids linked together by peptide bonds or modified peptide bonds. The term “polypeptide(s)” denotes both short chains, commonly called peptides, oligopeptides and oligomers, and longer chains, generally called proteins. The polypeptides may contain amino acids other than the 20 amino acids encoded by the gene. “Polypeptides” comprise those which are modified by natural processes, such as transformation and other post-translational modifications, but also by chemical modification techniques. Such modifications are well described in the scientific literature and are well known to persons skilled in this field. It will be appreciated that the same type of modification can be present to the same degree or to different degrees on several sites in a given polypeptide. Likewise, a given polypeptide may contain numerous types of modification. The modification may occur anywhere in a polypeptide, including the peptide backbone, the amino acid side chains and the amino or carboxylic ends. The modifications comprise, for example, acetylation, acylation, ADP-ribosylation, amidation, covalent binding of flavin, covalent binding of a fragment of heme, covalent binding of a nucleotide or nucleotide derivative, covalent binding of a lipid or lipid derivative, covalent binding of phosphotidylinositol, cross-linking, cyclization, formation of disulfide bonds, demethylation, formation of covalent transverse bonds, formation of cysteine, formation of pyroglutamate, formylation, gamma-carboxylation, formation of GPI anchors, hydroxylation, iodation, methylation, myristoylation, oxydation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, addition of amino acids to proteins via RNA transfer, such as arginylation, and ubiquitination. The polypeptides may be branched or cyclic, with or without branching. The cyclic, branched and unbranched polypeptides may result from post-translational natural processes and can also be manufactured by fully synthetic methods.

Preferably, the TSHR proteic acid sequence is at least 60% identical to the sequence SEQ ID NO: 1, for example 65%, 70%, 75%, 80%, 86%, 87%, 88%, 89%, 90%, 91%, 92%, 93%, 94%, more preferably at least 95% identical to the sequence SEQ ID NO: 1, for example 96%, 97%, 98%, 99%, even more preferably is identical to the sequence SEQ ID NO: 1.

Preferably, the TSHR nucleic acid sequence is at least 60% identical to the sequence SEQ ID NO: 2, for example 65%, 70%, 75%, 80%, 86%, 87%, 88%, 89%, 90%, 91%, 92%, 93%, 94%, more preferably at least 95% identical to the sequence SEQ ID NO: 2, for example 96%, 97%, 98%, 99%, even more preferably is identical to the sequence SEQ ID NO: 2.

Identity refers to a relationship between two or more polypeptide sequences or two or more polynucleotide sequences, as determined by the comparison of the sequences. In the art, “identity” also means the sequence genetic relationship between the polypeptide or polynucleotide sequences, depending on the case, as determined by the correspondence between the chains of these sequences. The “identity” and “similarity” can be easily calculated by known methods, including but not limited to those described in the following references (Computational Molecular Biology, Lesk A. M., ed., Oxford University Press, New York, 1988; Biocomputing: Informatics and genome Projects, Smith D. W., ed., Academic Press, New York. 1993; Computer Analysis of sequence Data, Part I, Griffin A. M., and Griffin H. G., eds., Humana Press. New jersey, 30 1994; sequence Analysis in Molecular Biology, von Heinje G., Academic Press, 1987; and sequence Analysis Primer, Gribskov M. and Devereux J., eds., M Stockton Press, New York, 1991; and Carillo H., and Lipman D., SIAM J. Applied Math., 48:1073 (1998)). The methods for determining the identity are designed to give the greatest correspondence between the sequences tested. Furthermore, the methods for determining the identity are coded in publicly accessible computer programs. Computer program methods for determining the identity between two sequences comprise, without limitation, the GCG software package (Devereux J. et al., Nucleic Acids Research 12(1): 387 (1984)), BLASTP, BLASTN, and FASTA (Altschul S. F. et al., J. Molec. Biol. 215: 403-410 (1990)). The BLAST X program is 12 publicly available from the NCBI and other sources (BLAST Manual, Altschul S. et al., NCBI NLM NUH Bethesda, MD 20894; Altschul S. et al., J. Mol Biol. 215: 403-410 (1990)).

By variant is meant a polynucleotide or a polypeptide which differs from a reference polynucleotide or polypeptide, respectively, but which retains essential properties. A typical variant of a polynucleotide differs by its nucleotide sequence from another reference polynucleotide. The modifications of the nucleotide sequence of the variant may or may not modify the amino acid sequence of a polypeptide encoded by the reference polynucleotide. The nucleotide changes can lead to substitutions, additions, deletions, fusions, and/or truncations of amino acids in the polypeptide encoded by the reference sequence, as discussed below. A typical variant of a polypeptide differs in amino acid sequence from another reference polypeptide.

In general, the differences are limited so that the sequences of the reference polypeptide and of the variant are overall very similar and, in numerous regions, identical. A variant polypeptide and a reference polypeptide may differ in the amino acid sequence by one or more substitutions, additions, and/or deletions in any combination. A substituted or inserted amino acid residue may or may not be a residue encoded by the genetic code.

The present invention also comprises variants of each of the polypeptides of the invention, that is to say polypeptides which vary from references by conservative substitutions of amino acids, by which a residue is replaced by another having similar characteristics. Typical conservative amino acid substitutions are from Ala, Val, Leu and IIe; from Ser and Thr; from the acid residues, Asp and Glu; from Asn and Gln; and from the basic residues, Lys and Arg; or the aromatic residues, Phe and Tyr. These conservative mutations comprise mutations that exchange an amino acid for another from one of the following groups:
1. Small aliphatic residues, non-polar or slightly polar: Ala, Ser, Thr, Pro, and Gly;
2. Polar residues, negatively charged and their amides: Asp, Asn, Glu and Gln;
3. Polar residues, positively charged: His, Arg and Lys;
4. Large non-polar aliphatic residues: Met, Leu, IIe, Val and Cys; and
5. Aromatic residues: Phe, Tyr and Trp.

These conservative variations can also include the following:

| Original residues | Variations |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

Preferably, the variants in which several, for example 5-10, 1-5, 1-3, 1-2 or 1 amino acid are substituted, deleted or added in any combination. A variant of a polynucleotide or of a polypeptide may be of natural origin, such as an allelic variant, or it may be a variant that is not known to be of natural origin. The non-natural variants of polynucleotides and polypeptides can be obtained by mutagenesis techniques, by direct synthesis and by other recombination methods known to a person skilled in the art.

As mentioned above, in addition to the GC content and/or number of ARF, the optimization of the sequence may also comprise a decrease in the number of CpG islands in the sequence and/or a decrease in the number of splicing donor and acceptor sites.

Of course, as is well known to a person skilled in the art, the optimization of the sequence is an equilibrium between all these parameters, which means that a sequence can be considered as optimized if at least one of the above parameters is improved whereas one or more of the other parameters are not, as long as the optimized sequence leads to an improvement of the transgene, such as an improvement in the expression and/or a decrease in the immune response to the transgene in vivo.

Preferably, the transgene of interest codes for a human TSHR, and the nucleic acid sequence coding for the human TSHR protein comprises an optimized sequence identical to the sequence SEQ ID NO: 2.

The term “liposome” is intended to mean an artificial vesicle formed by concentric lipid bilayers, trapping the aqueous compartments between them. It is obtained from a large variety of amphiphilic lipids, most often phospholipids. When such compounds are placed in the presence of an excess of aqueous solution, they are organized so as to minimize the interactions between their hydrocarbon chains and water.

The genetic activator preferably used according to the invention is an adenoviral vector, more preferably an adeno-associated virus (AAV), even more preferably an AAV8 or an AAV9.

Another subject matter of the invention relates to a combination of a pharmacological activator of the TSHR signaling pathway in muscle stem cells (satellite cells) and of a genetic activator of the TSHR signaling pathway in muscle stem cells (satellite cells) for use in preserving and regenerating muscle structure and function by blocking senescence, preferably for preventing and/or treating myopathies, more preferably DMD or else for preventing and/or treating sarcopenia.

According to a particularly preferred embodiment of the invention, in the combination used according to the invention, the pharmacological activator is forskolin and the genetic activator is an adeno-associated virus (AAV) comprising a thyroid-stimulating hormone receptor (TSHR) protein sequence of SEQ ID NO: 1 or a functional variant having a protein sequence at least 60% identical to the sequence SEQ ID NO: 1, or a TSHR nucleotide sequence of SEQ ID NO: 2 or a functional variant having a nucleotide sequence at least 60% identical to the sequence SEQ ID NO: 2, more preferably an AAV8 or an AAV9.

Another object of the invention relates to an adenoviral vector comprising a thyroid-stimulating hormone receptor (TSHR) protein sequence of SEQ ID NO: 1 or a functional variant having a protein sequence at least 60% identical to the sequence SEQ ID NO: 1, or a TSHR nucleotide sequence of SEQ ID NO: 2 or a functional variant having a nucleotide sequence at least 60% identical to the sequence SEQ ID NO: 2 for use as a medicinal product.

The adenoviral vector preferably used according to the invention is an adeno-associated virus (AAV), preferably an AAV8 or an AAV9.

The adenoviral vector is preferably used according to the invention for preserving and regenerating muscle structure and function by blocking senescence, more preferably for preventing and/or treating myopathies, even more preferably DMD.

According to another embodiment of the invention, the adenoviral vector is preferably used according to the invention for preventing and/or treating sarcopenia.

### EXAMPLES

The data analysis was carried out using the GraphPad Prism software, version 6.0. The data are expressed as average±standard deviation.

### Example 1: Generating R-DMDdel52 Rats

The rat R-DMDdel52 model is generated by the deletion of 188 bp (base pairs) and the generation of a premature stop codon at exon 52 of the dystrophin gene (DMD) to produce a loss of function of the allele responsible for dystrophin production.

The sequence of the rat exon 52 (ENSRNOE00000518152) and the intronic sequences around were selected in order to identify the guide RNA sequences (sgRNAs) by the BWA algorithm for genomic editing by the CRISPR method.

After validation in vitro, two sgRNAs 3′ and 5′ were selected and microinjected into the oocytes of Sprague Dawley rats.

The animals F0 were screened by genotyping by junction PCR. Genotyping is carried out on DNA extracted from biopsies of the tail or of the ear.

The PCR mixture was prepared with the Dream Taq 10× buffer (ThermoFisher Scientific, Cat #B65), 0.25 mM of dNTP (10 mM), 0.3 μM of the sense primer ((5′-CTAACGCATTTAAAATATGCTGTCA-3′), 0.3 μM of the antisense primer (5′-GTTGGCTTAGCTCAACAACCAAGAT-3′, 100 μM), 0.03 U/μl DreamTaq and approximately 150 ng of DNA. The PCR was carried out using a Thermal Cycler 2720 with the following heat program: initial denaturation at 95° C. for 5 minutes, 40 cycles of denaturation at 95° C. for 10 seconds, hybridization of the primers at 60° C. for 30 seconds and elongation at 72° C. for 45 seconds, and final elongation at 72° C. for 5 minutes.

The deletion of exon 52 was detected on a 2% agarose gel.

The rats were housed in an installation free of pathogens with cycles of 12 hours of light and 12 hours of darkness, in accordance with European Directive 2010/63/EU.

Only male rats were used for the experiments since the mutation is carried on chromosome X.

The animals were handled in accordance with national and European directives, and the protocols were approved by the Ethics Committee of the French Ministry (APAFIS #25606-202005311746599).

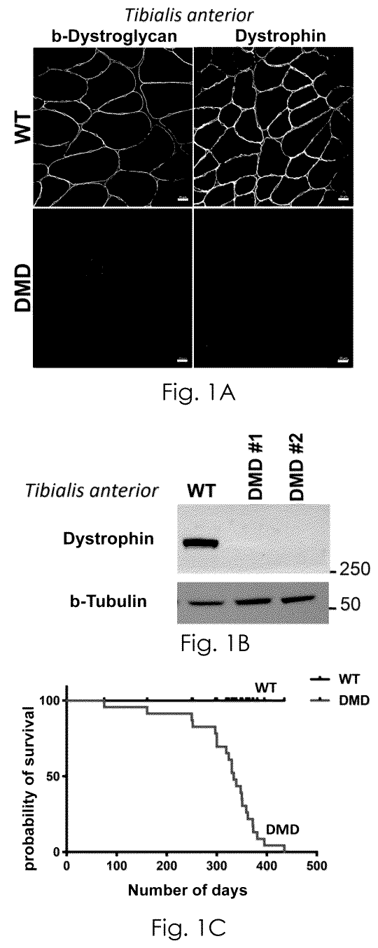

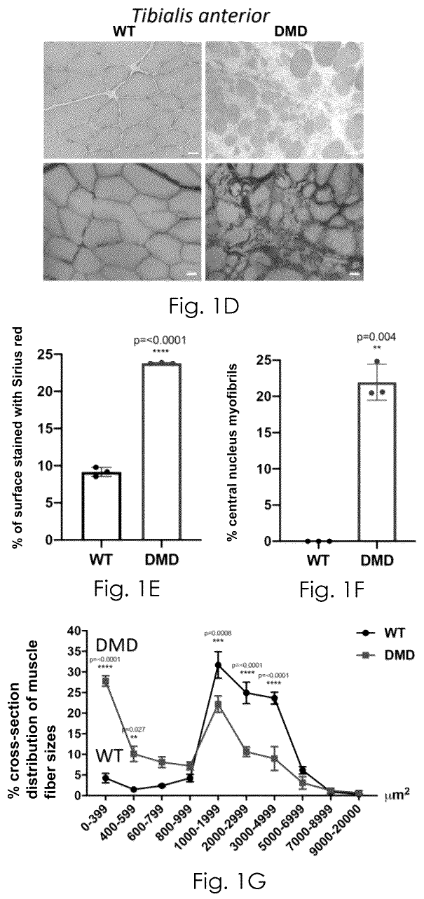

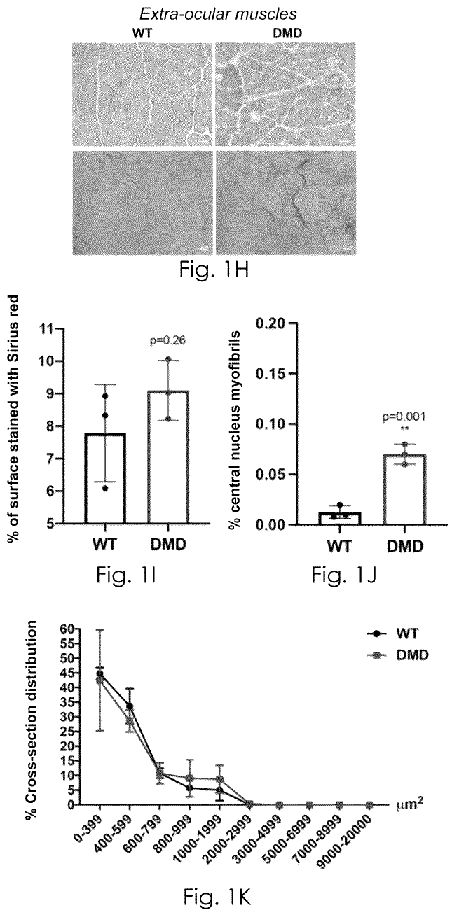

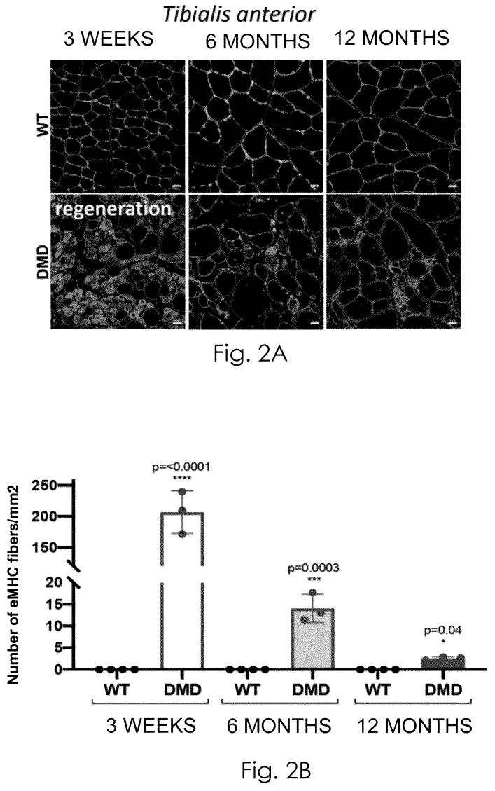

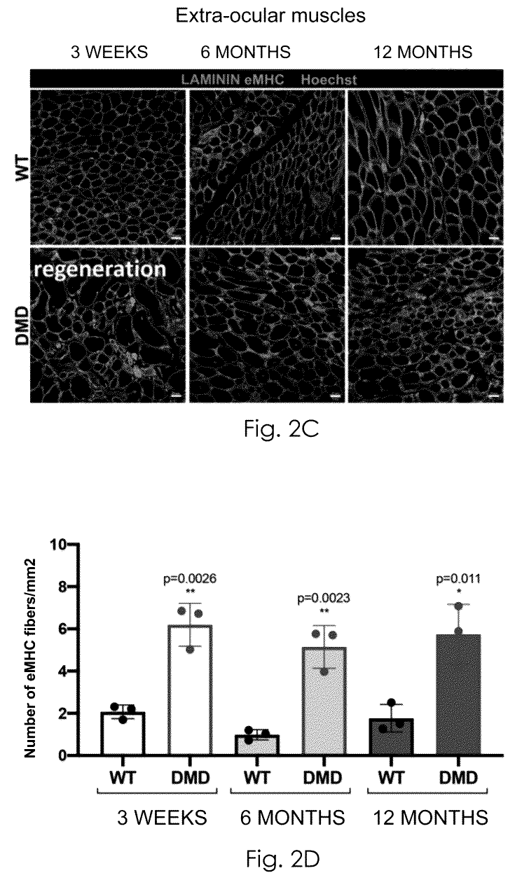

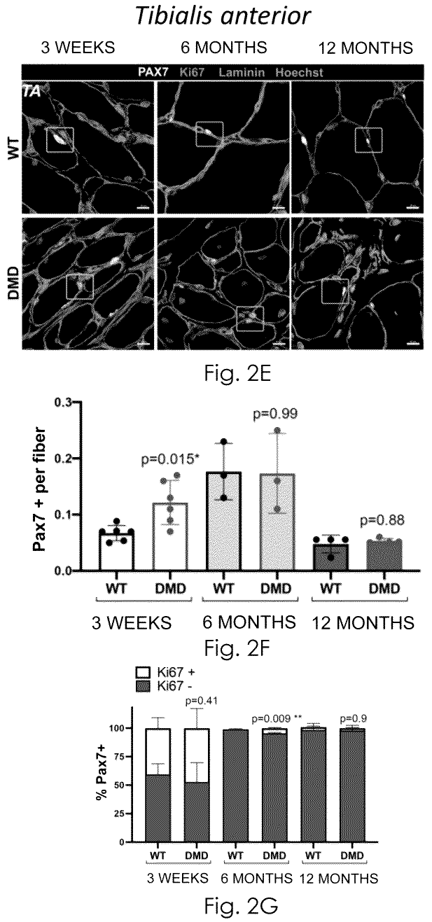

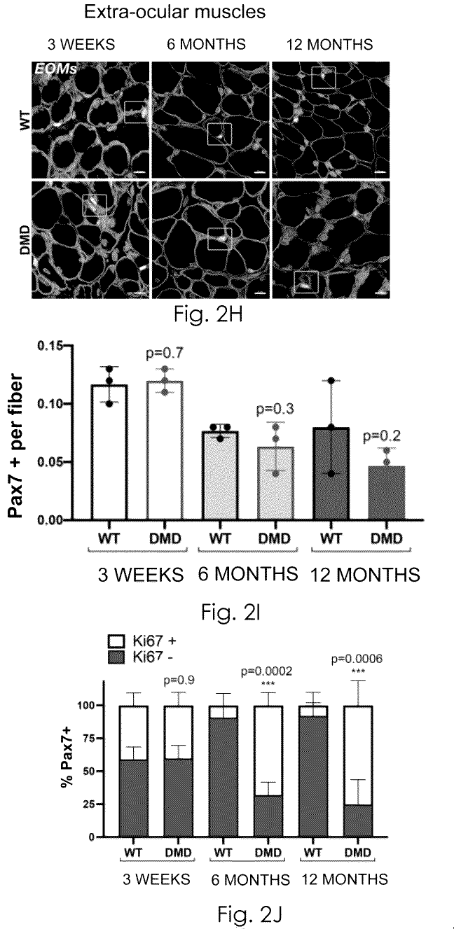

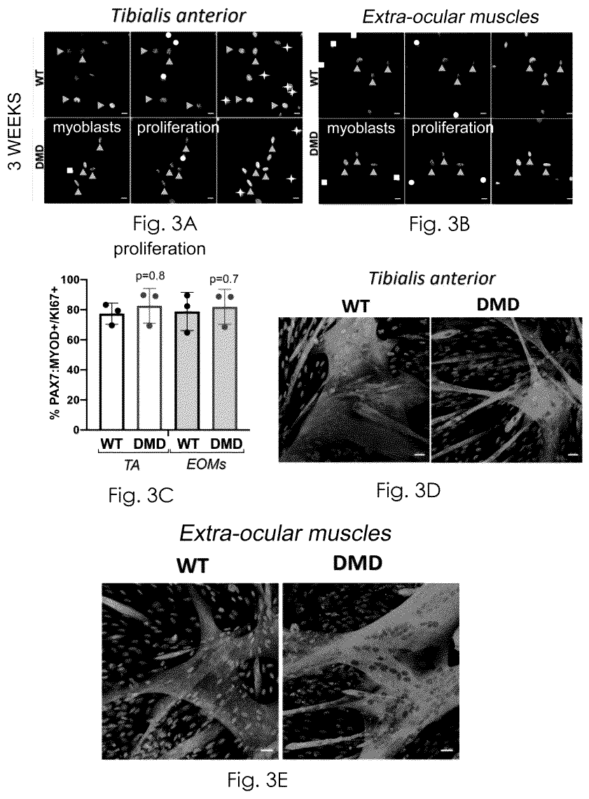

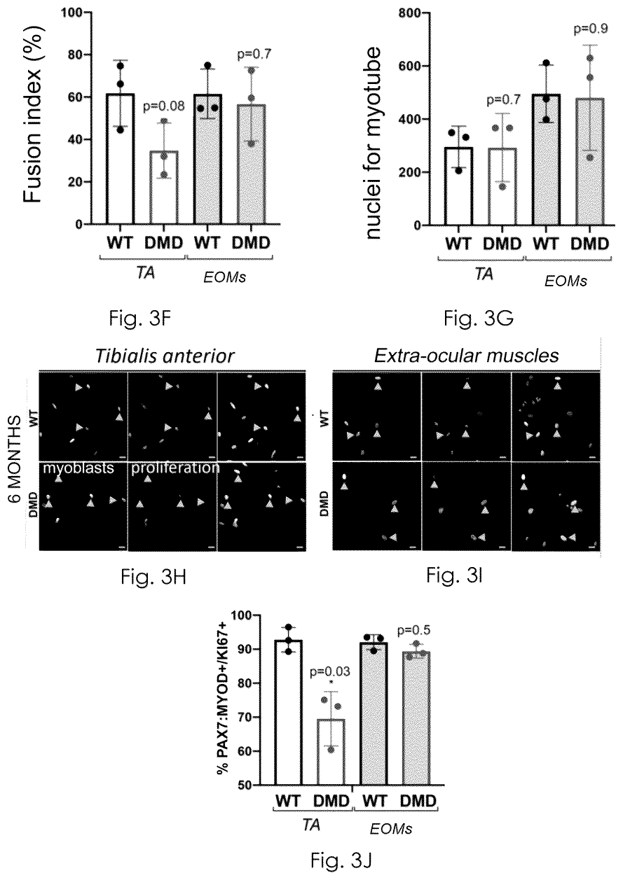

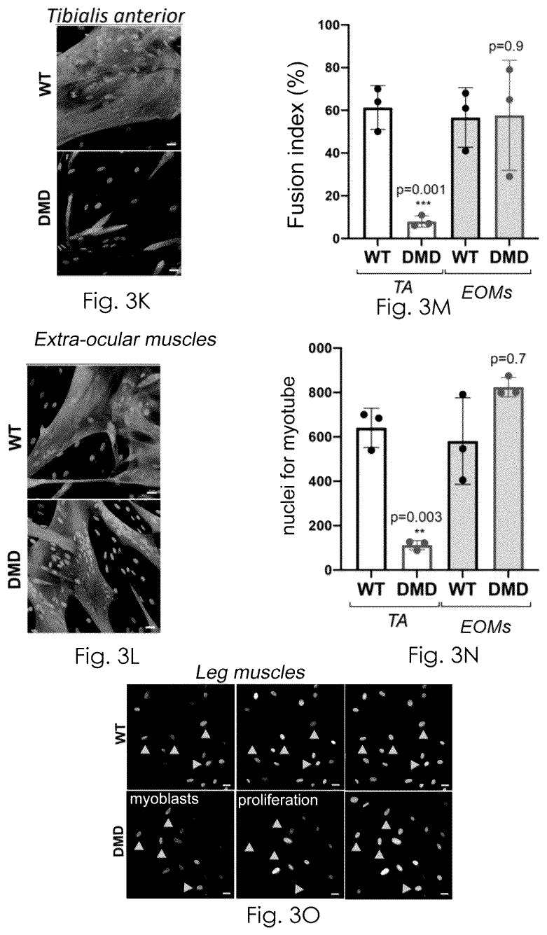

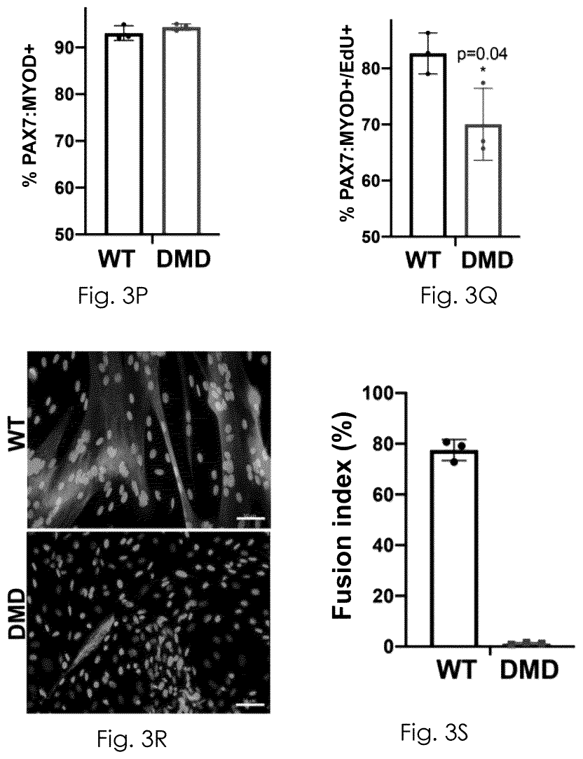

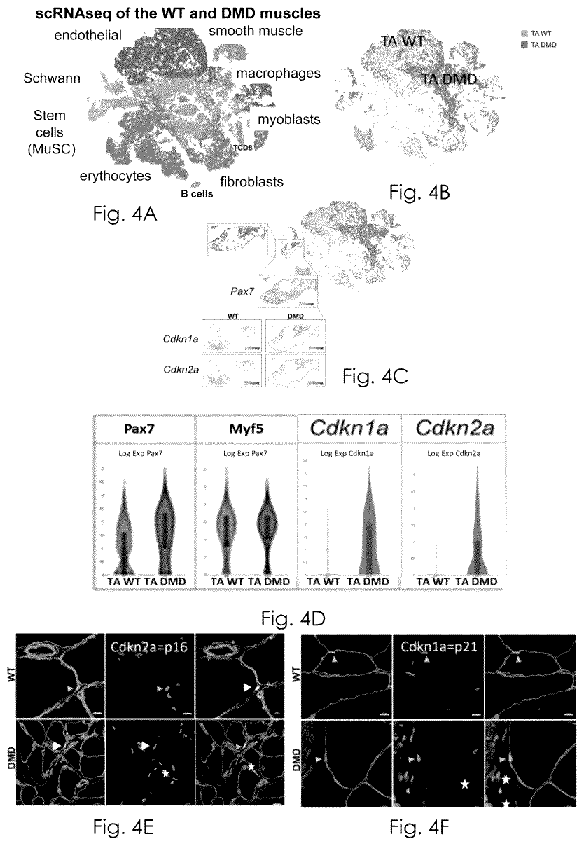

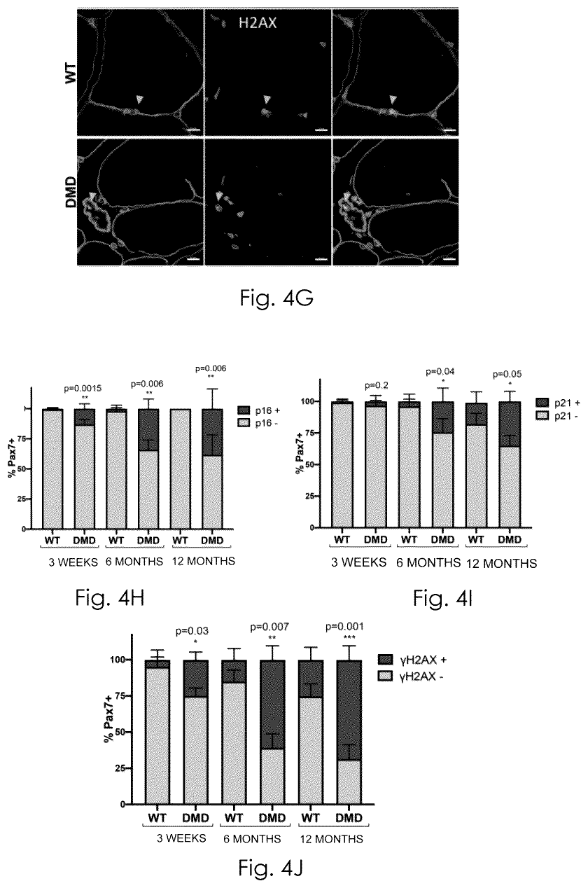

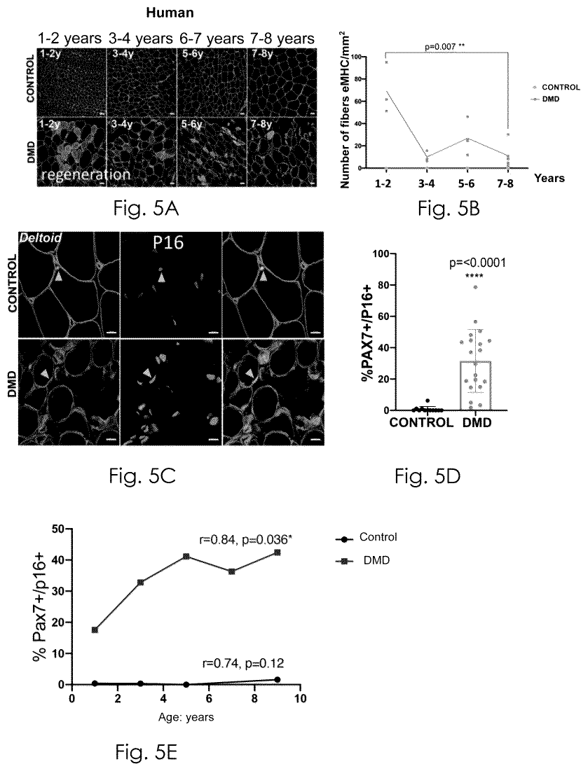

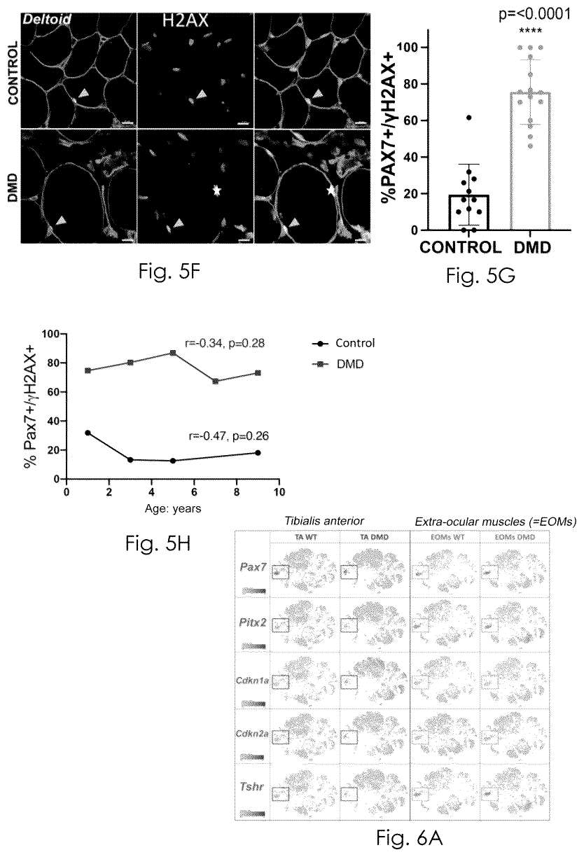

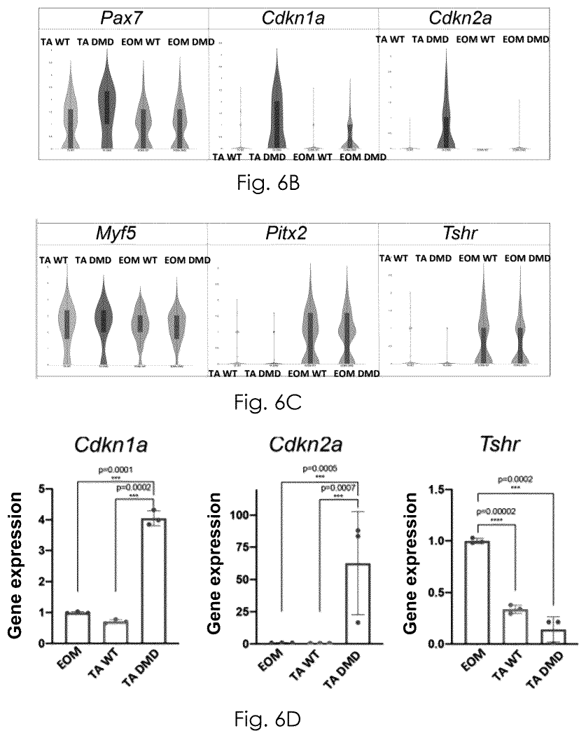

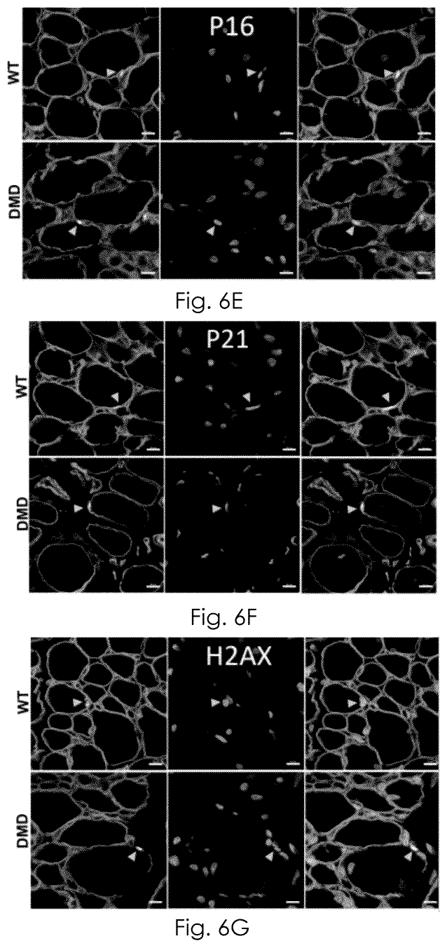

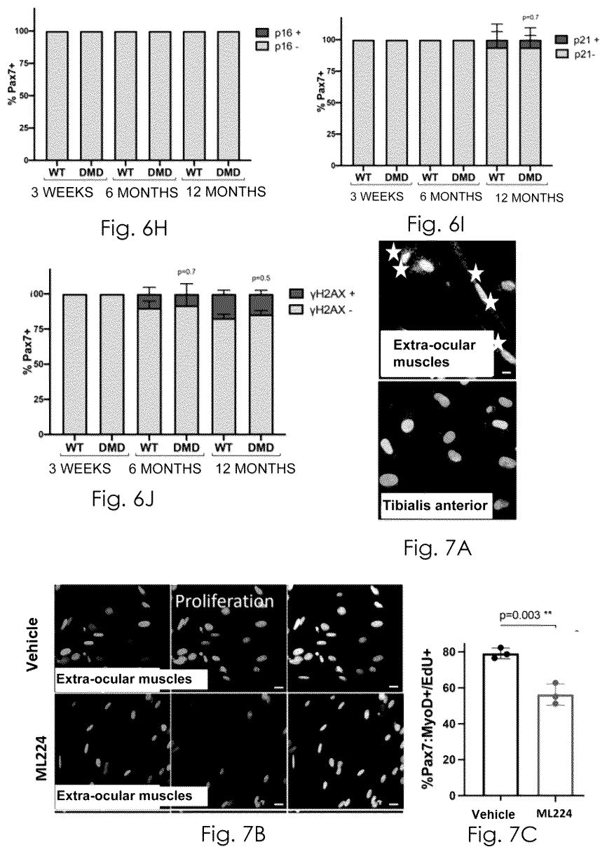

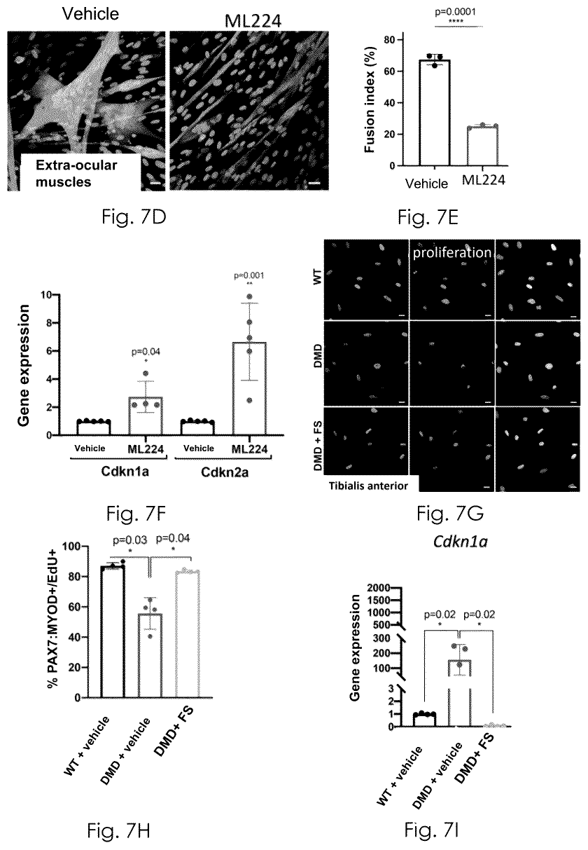

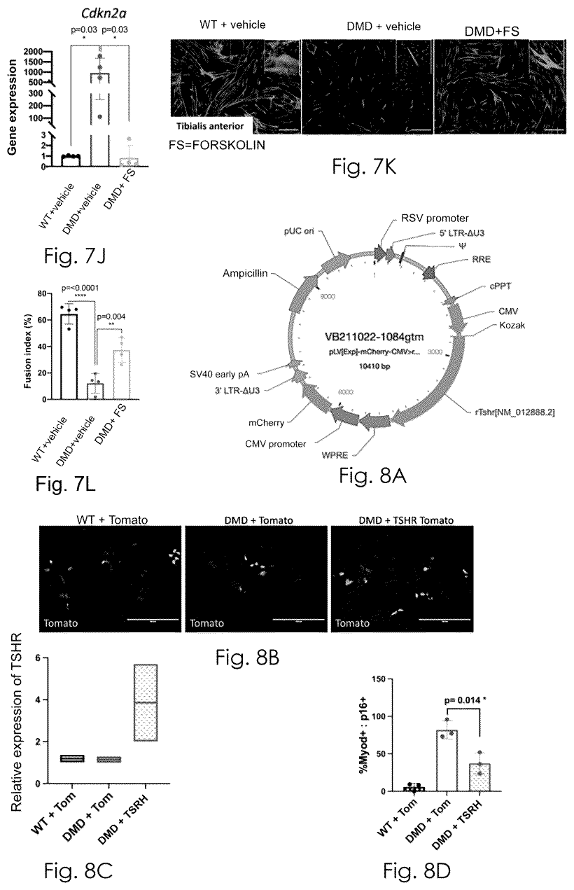

The expression of the dystrophin was then evaluated by immunofluorescence (FIG. 1A) and western blot (FIG. 1B) in the muscles of the WT and R-DMDdel52 rats.

The western blot was performed using cryosections of frozen muscle which were homogenized using a dounce homogenizer in a lysis buffer containing 50 mM Tris-HCl, pH 7.4, 100 mM NaCl, 0.5% NP40 and a Halt protease inhibitor cocktail (Pierce).

The samples were then centrifuged for 5 minutes at 1500 g and denatured at room temperature for 30 minutes with Laemmli buffer. The protein concentration was determined by the Bradford test (Pierce). The proteins were separated by electrophoresis (Nu-PAGE 4-12% Bis-Tris gel; Life Technologies) then transferred to nitrocellulose membranes (GE Healthcare) et labeled with primary antibodies and secondary antibodies coupled horseradish peroxidase. The primary antibody used is dystrophin (DYS2, Leica) and alpha-tubulin (Sigma, T5168). The signals were displayed with the chemiluminescent SuperSignal West Pico (Pierce) substrate. The images were acquired with Chemidoc MP (Biorad).

This made it possible to determine that the dystrophin is not expressed in the R-DMDdel52 rats compared to the WT rats. (FIG. 1A-B).

A survival curve was established in order to compare the probability of survival between the WT and R-DMDdel52 rats as a function of the number of days. This made it possible to show that the R-DMDdel52 rats die after 14 months of life, unlike the WT rats.

In addition, the R-DMDdel52 rats have a body mass that is much lower than that of the WT controls starting from the age of 3 months, with a large decrease in the body mass index and the length of the body at the age of 12 months (Table 1).

In agreement with the progressive loss of muscle mass, a reduction in the weight of the Tibialis anterior (TA) and soleus was observed in the 12-month-old R-DMDdel52 rats compared with the WT rats.

A histological examination was carried out by hematoxylin and eosin (H&E) and staining with Sirius red at the tibialis anterior in the R-DMDdel52 rats compared to the WT rats. (FIG. 1D-G)

For hematoxylin and eosin staining, the TA muscles are isolated in 12-month-old WT and R-DMDdel52 rats. After isolation, the muscles are immediately frozen in isopentane cooled in liquid nitrogen and cut at 7 μm.

The slides were immersed in Mayer hematoxylin (Sigma-Aldrich) for 3 minutes and after rinsing in deionized water, they are immersed in a solution of lithium carbonate for 3 seconds, then incubated in eosin for 30 seconds. After washing with distilled water, the slides are immersed in 50% and 70% ethanol, then equilibrated in 95% and 100% ethanol.

The slides were mounted with Eurokit after having been soaked in xylene.

For Sirius red staining, slides are thawed for 20 minutes at room temperature (RT) and immersed in 90% EtOH for 2 minutes before starting the staining, by incubating the samples for 25 minutes in a solution of Picro sirius red (Sigma-Aldrich). The slides are then copiously washed with water and dehydrated in 100% ethanol.

After clarifying with xylene, the slides were mounted in Eurokit.

This made it possible to demonstrate that the muscles of the R-DMDdel52 rats exhibit signs of severe DMD pathology with the presence of endomysial fibrosis, altered fiber morphology and the invasion of inflammatory cells. (FIG. 1D, upper panels).

Indeed, severe fibrotic deposits are confirmed by the staining with Sirius red. (FIG. 1D, lower panels). The extent of fibrosis is quantified by measuring the percentage of the area occupied by the connective tissue, showing statistically significant differences between the WT and R-DMDdel52 rats at the tibialis anterior (FIG. 1E). Indeed, the R-DMDdel52 rats have approximately 2.5 times more fibrosis than the WT rats.

The presence of central nuclei is also evaluated, as a pathological marker of a muscle undergoing a degeneration/regeneration event. The muscle in R-DMDdel52 had a considerable number of central nucleus fibers relative to the controls. (FIG. 1F)

Finally, the presence of atrophic fibers is confirmed, with a cross-sectional area of the reduced muscle fibers, which contribute to the muscule wasting (Shin, Jonghyun et al. “Wasting mechanisms in muscular dystrophy.” The international journal of biochemistry & cell biology vol. 45,10 (2013): 2266-79. doi:10.1016/j.biocel.2013.05.001). (FIG. 1G)

The extra-ocular muscles (EOM) were also analyzed, since they are selectively spared in Duchenne muscular dystrophy (Kaminski, H J et al. “Extraocular muscles are spared in advanced Duchenne dystrophy.” Annals of neurology vol. 32,4 (1992): 586-8. doi:10.1002/ana.4103204181992). EOM are complex striated muscles which have distinct biochemical, mechanical and physiological properties compared to their homologs of the limbs (Bondi, A Y, and D J Chiarardini. “Morphologic and electrophysiologic identification of multiply innervated fibers in rat extraocular muscles.” Investigative ophthalmology & visual science vol. 24,4 (1983): 516-9., Jacoby et al., 1990, Kono et al., 2005, Fraterman et al., 2007). Stainings with hematoxylin/eosin and Sirius red confirmed the preservation of the EOM ISOLATED FROM 12-MONTH-OLD R-DMDdel52 ANIMALS (FIG. H-K), WITHOUT ANY SIGN of significant inflammation or fibrosis relative to the controls (FIG. 1H-I).

It is interesting to note that the EOM R-DMDdel52 had a high number of central nucleus myofibers relative to the WT muscles (FIG. 1J), but to a lesser extent relative to the TA (FIG. 1F). Furthermore, the analysis of the size of the fibers did not show significant variation between the EOM of the 12-month-old WT and R-DMDdel52 rats (FIG. 1K).

These results show that the R-DMDdel52 exhibit a progressive and severe muscular dystrophy characterized by well-described characteristics of the physiopathology of DMD, including the fact that the extra-ocular muscles are specifically not affected by the disease.

TABLE 1

| | 3 weeks | | 6 months | | 12 months | |
|---|---|---|---|---|---|---|
| Parameter | WT | DMD | WT | DMD | WT | DMD |
| Body mass (g) | 96.9 ± 21.4 | 76.0 ± 3.4 | 621.3 ± 38.4 | 547.2 ± 60.0* | 731.1 ± 63.3 | 337.7 ± 88.9*** |
| Body mass index (g/cm²) | 0.39 ± 0.03 | 0.34 ± 0.03 | 0.70 ± 0.06 | 0.63 ± 0.04 | 0.74 ± 0.04 | 0.34 ± 0.22** |
| Body size (cm) | 15.7 ± 1.2 | 14.9 ± 0.6 | 29.1 ± 1.0 | 29.0 ± 1.3 | 30.5 ± 1.1 | 29.6 ± 0.7 |
| Tibialis anterior (g) | 0.16 ± 0.03 | 0.12 ± 0.01 | 1.21 ± 0.34 | 0.97 ± 0.22 | 1.15 ± 0.05 | 0.55 ± 0.21** |
| Heart (g) | 0.47 ± 0.06 | 0.47 ± 0.05 | 1.77 ± 0.10 | 1.84 ± 0.48 | 1.86 ± 0.11 | 2.20 ± 0.45§ |
| Soleus (g) | 0.054 ± 0.03 | 0.035 ± 0.02 | 0.39 ± 0.03 | 0.25 ± 0.13 | 0.35 ± 0.10 | 0.15 ± 0.06* |

The p values are calculated with reference to the WT
*p < 0.05
**p < 0.01
***p < 0.001
§p = 0.07

### Example 2: The Extra-Ocular Muscles do not have Progressive Muscle Regeneration Defects in the DMD Model of the Rat

The lesions of the muscle tissue lead to the necrosis of the muscle fibers which trigger muscle regeneration by activating the pool of quiescent muscle satellite cells located near muscle fibers.

The temporal progression of muscle regeneration in the tibialis anterior of the R-DMDdel52 rats was evaluated by using immunofluorescence for embryonic myosin heavy chain (eMHC), a specific marker of myofibers in development and regeneration on histological sections of skeletal muscles.

Immunohistochemistry is thus carried out on cryosections: The fresh frozen muscles were sectioned with the Leica CM3050S cryostat (Leica Biosystems) at 7 μm thickness on Super Frost Plus slides (Thermo scientific, 10149870) and stored at −80° C.

The slides were thawed for about 20 minutes at room temperature, rehydrated for 5 minutes in 1×PBS and then fixed with 4% PFA in 1×PBS for 10 minutes at 4° C. After washing in 1×PBS, the slides were placed in a cold solution of acetone and methanol (1:1) for 6 minutes at −20° C., then incubated with 10% BSA for 1 hour of blockage. The primary antibodies were added at 1% BSA and incubated overnight (O/N) at 4° C.

The following primary antibodies were used: Pax7 (Santa Cruz Biotechnology, sc-81648), ki67 (Abcam, sp6 ab16667), Cdkn2a (Abcam Anti-CDKN2A/p16INK4a, Ab108349), Cdkn1a (Thermofisher, bs-10129R) and YH2AX (Abcam, ab11174). After repeated washes, the slides were incubated with Alexa Fluor secondary antibodies for 45 minutes at room temperature. The staining of the laminin was carried out after incubation of the secondary antibodies, for 1 h at 37° C. using a conjugated antibody (NB300-144AF647).

The nuclei were stained with Hoechst (Sigma-Aldrich, B2261). The fluorescence was analyzed with an LSM800 confocal apparatus. The immunolabeling of the embryonic myosin heavy chain (eMHC), dystrophin and 3-dystroglycan was carried out without binding and with permeabilization in 0.5% Triton in 1×PBS for 5 minutes. The eMHC (sc-53091), dystrophin (Leica, DYS2-CE), β-dystroglycan (Leica, B-DG-CE) and laminin (Sigma-Aldrich, L9393) were incubated in fresh air.

The incubation of the secondary antibodies and the staining of the nuclei were carried out as described above. The fluorescence was analyzed with an LSM800 confocal apparatus.

A large number of eMHC positive myofibers in the TA of the R-DMDdel52 rats aged 3 weeks is observed, whereas they were completely absent in the WT muscles (FIG. 2A).

A large decrease in the number of fibers in regeneration was observed over time (FIG. 2A-B), indicating a progressive reduction in muscle regeneration during the progression of muscular dystrophy.

On the other hand, although it is slightly increased in the R-DMDdel52 rats relative to the WT rats, the regeneration process is stable and limited during the pathological progression in the extra-ocular muscles.

These data suggest that EOMs have improved regeneration performance compared to other muscles.

Since the ability to regenerate skeletal muscles is linked to the satellite cells, the decision was made to evaluate the number and the myocardial status of the MSCs in vivo.

Co-immunolabeling is carried out for Pax7 and Ki67, a proliferation and activation marker, on histological sections of skeletal muscles isolated from TA and EOM of WT and R-DMDdel52 rats.

As shown in FIGS. 2E and 2H, no difference is observed in the number of MSCs per fiber between the WT and R-DMDdel52 TA or EOM muscles (quantified in FIGS. 2F and 2I), which indicates that the MSCs are not lost during the pathological progression of DMD.

The status of the cell cycle of the MSCs is then evaluated in the WT and R-DMDdel52 rats. It can be said that in the TA muscle of the WT and R-DMDdel52 rats, the MSCs only divide at early stages (3 weeks), while remaining arrested in their cell cycle at subsequent stages of the pathology, including in the pathological context of DMD.

On the other hand, a large part of the MSCs analyzed on EOM histological sections expressed the proliferation marker, Ki67, both in the WT and R-DMDdel52 samples and at all the stages examined, including in the 12-month-old WT and dystrophic rats (FIG. 2H-J).

This suggests that EOM MSCs are highly active in a WT context and retain their regeneration capacity in R-DMDdel52 animals.

### Example 3: R-DMDdel52 TA MSCs have an Impaired Myogenic Potential In Vitro

In order to investigate the properties of WT and R-DMDdel52 MSCs, the satellite cells of the Tibialis anterior and extra-ocular muscles were isolated and their proliferative capacity was evaluated in culture.

For this, the cells were fixed with 4% PFA for 10 minutes at 4° C. and permeabilized with 0.5% Triton in 1×PBS for 6 minutes. After blocking with 10% BSA for 30 minutes at room temperature, the cells were incubated O/N at 4° C. with the following primary antibodies: Pax7 (Santa Cruz Biotechnology, sc-81648), MyoD (Dako, clone 5.8A, M3512), ki67 (Abcam, sp6 ab16667), sarcomeric myosin heavy chain (MF20, DSHB, AB_2147781).

Significant differences were not observed in the proliferative properties of the MSCs isolated from the TA or the EOMs of the 3-week-old WT and R-DMDdel52 rats (FIG. 3A-C).

Furthermore, the cultures of myoblasts derived from WT and R-DMDdel52 MSC isolated from controls of the TA and EOM muscles did not exhibit significant differences in myogenic differentiation (FIG. 3D-G).

It can be seen that the MSCs derived from the TA of 6-month-old R-DMDdel52 rats exhibit a decrease in the percentage of MyoD-positive myoblasts co-expressing Ki67 relative to the WT control cells (FIG. 3I-J).

However, there is no significant difference in proliferation in the myoblasts isolated from 6-month-old R-DMDdel52 EOM relative to the WT EOM, which suggests that the MSCs of the R-DMDdel52 extra-ocular muscles retain their proliferation capacity ex vivo (FIG. 3I-J).

Next, myogenic differentiation was evaluated on cultured cells isolated from the TA muscles of 6-month-old WT and R-DDdel52 rats.

Whereas differentiation of the myogenic cell cultures isolated from the EOM of 6-month-old R-DMDdel52 was practically indistinguishable from that of the controls, a large differentiation deficiency is observed in the cultures of R-DMDdel52 cells isolated from the TA of the same rats. (FIG. 3K-L)

The quantification showed a significant reduction in the fusion index as well as the number of nuclei per myotube per mm² in the cultured R-DMDdel52 TA cells relative to the WT controls. (FIG. 3M-N)

Conversely, no significant differentiation defect was observed in the myoblasts isolated from the EOMs of dystrophic rats and of WT rats aged 6 months. (FIG. 3M-N)

In order to evaluate whether the proliferation and differentiation defects observed during the culture of cells isolated from R-DMDdel52 TA muscles are cell-autonomous, the MSCs are purified from limb muscles by FACS sorting with CD45, CD31, Ter119 as exclusion markers and α7-integrin as a positive selection surface marker (FIG. S2A), allowing the culture of pure myoblasts derived from MSC (FIG. 3 O-P).

FACS sorting is carried out by the following method: After dissociation of the cells, the cells isolated from the posterior limb were incubated with CD45-PE-Cy7 (BD Pharmingen, 552848), Ter119-PE-Cy7 (BD Pharmingen, 557853), CD31-PE-Cy7 (BD Pharmingen, 561410), α7-integrin-FITC (CliniSciences, C179570-100) for 45 minutes at 4° C. After washing with HBSS supplemented with DNase (Sigma-Aldrich, DN25), the cells are incubated with the vital dye 7-AAD (BD Pharmingen, 559925) and sorted with the BD FacsAria device. The population of MSCs was identified as CD45:Ter119:CD31-negative and α7-integrin-positive.

A decrease in the proliferative potential is observed in sorted R-DMDdel52 cells, as shown by the lower number of myoblasts (Pax7±:MyoD±) labeled with EdU (FIG. 3Q) in cultured cells originating from dystrophic limb muscles.

The sorted muscle satellite cells R-DMDdel52 also have large deficiencies in myogenic differentiation with the formation of small myotubes with fewer nuclei compared to the WT cells (FIG. 3R-S).

Taken together, these data demonstrate that the proliferation and differentiation are impaired in the MSCs isolated from the R-DMDdel52 limb muscles, whereas the MSCs of the dystrophic EOMs retain their potential.

### Example 4: The Satellite Cells of the R-DMDdel52 Tibialis Anterior Muscle Express Senescence Markers

In order to study the molecular and cellular changes occurring in the muscles of dystrophic rats, a scRNA-seq is carried out from the TA muscles of 12-month-old R-DMDdel52 and WT rats aged 12 months.

Indeed, the cells of the TA and extra-ocular muscles are extracted from WT and R-DMDdel52 rats aged 12 months and immediately digested with 3 U/ml of Dispase II (Roche, 4942078001) and 0.5 u/ml of collagenase A (Roche, 10103586001) for 20 minutes in HBSS at 37° C. with slight stirring.

The cell suspension was passed through cell filters and centrifuged at 600 g/minute for 5 minutes, at 4° C. The supernatant was removed and the cell pellet was resuspended in 20% fetal bovine serum (Cat #S1810-500 Dominique Dutscher) in DMEM (Cat #41966-029 Gibco) supplemented by 25 ng/mL of bFGF (RP4037, Sigma-Aldrich) with 100 mg/mL of penicillin/streptomycin (354234, Life Sciences) covered with matrigel (Sarstedt, #94.6140.802). The cells were grown for 48 hours to evaluate the proliferation. After 48 h of proliferation, the cells were cultured for 5 days in 5% fetal bovine serum to induce differentiation.

After dissociation, the cells were filtered in 100 and 70 μm cell strainers and sorted by FACS after incubation in DAPI (D9542, Sigma-Aldrich) to obtain a specific cell suspension eliminating dead cells and debris.

The snRNA-seq is performed using the Chromium Next GEM Single Cell 3′Gene Expression V3 kit (10× Genomics) by following the manufacturing protocol. The sequencing was carried out on the Illumina NexSeq 550 system with High Output Kit v2.5 (75 cycles, Illumina 20024906).

The cells were filtered based on sample readings, the mitochondrial and ribosomal RNA content and the predicted doublets to obtain an average of 6247 cells per sample (Table 2). The identity of the cells was evaluated on the basis of the expression of well established markers and the data were visualized by t-distributed stochastic neighbor embedding (Jamieson, Andrew R et al. “Exploring nonlinear feature space dimension reduction and data representation in breast Cadx with Laplacian eigenmaps and t-SNE.” Medical physics vol. 37,1 (2010): 339-51. doi:10.1118/1.3267037) (t-SNE; FIG. 4A). A total of 9 major groups of cells residing in the muscles is identified. (FIG. S2A).

WT and R-DMDdel52 cells are labeled on the t-SNE map, demonstrating transcriptomic level disease changes (FIG. 4B). These changes were obvious for the MSCs which exhibited distinct gene expression signatures between the WT and R-DMDdel52 samples (FIG. 4C).

Identification of the genes differentially expressed in the group of MSCs is thus studied.

The specific induction of two growth and senescence stop markers in the R-DMDdel52 MSCs relative to the controls is identified: cyclin-dependent kinase Cdkn1a (p21) and Cdkn2a inhibitors (pl6Ink4a, Cdkn2a) (Hernandz-Segura, Alejandra et al. “Hallmarks of Cellular Senescence.” Trends in cell biology vol. 28,6 (2018): 436-453. doi:10.1016/j.tcb.2018.02.001) (FIG. 4C-D).

The expression of Cdkn1a and Cdkn2a is observed in MSCs on the protein level by co-immunolabeling with Pax7 on muscle sections isolated from 6-month-old R-DMDdel52 and WT rats.

The presence of the phosphorylated form of histone H2A gamma X (YH2AX), a marker of DNA damage induced in senescent cells (Wang, Zhong et al. “RNA-Seq: a revolutionary tool for transcriptomics.” Nature reviews. Genetics vol. 10,1 (2009): 57-63. doi:10.1038/nrg2484; Bernadotte, Alexandra et al. “Markers of cellular senescence. Telomere shortening as a marker of cellular senescence.” Aging vol. 8,1 (2016): 3-11. doi:10.18632/aging. 100871; Dungan, Cory M et al. “In vivo analysis of YH2AX+ cells in skeletal muscle from aged and obese humans.” FASEB journal: official publication of the Federation of American Societies for Experimental Biology vol. 34,5 (2020): 7018-7035. do:10.1096/fj.202000111RR) in the MSCs of dystrophic rats relative to the WT rats (FIG. 4F-H) is also observed.

In a striking manner, the senescence signature of the MSCs in vivo (Cdkn2a, Cdkn1a, YH2AX) was associated with the progression of the disease in R-DMDdel52 (FIG. 4H-J).

These data suggest that the R-DMDdel52 TA MSCs progressively acquire a senescence signature during the progression of the disease, which is consistent with the progressive reduction of the regenerator potential and the phenotypes observed in the ex vivo cultures of MSCs.

TABLE 2

| | Estimation of | Average | Median | | | Saturation |
|---|---|---|---|---|---|---|
| | the number of | readings | genes | Number of | Valid | of the |
| | cells | per cell | per cell | readings | barcodes | sequencing |
| WT TA | 3,550 | 133,326 | 695 | 473,307,648 | 97.7% | 92.7% |
| DMD TA | 8,944 | 56,705 | 629 | 507,170,789 | 97.1% | 86.5% |
| WT EOM | 1,917 | 234,365 | 1,012 | 449,279,284 | 98.3% | 93.1% |
| DMD EOM | 3,093 | 144,443 | 1,350 | 446,762,721 | 97.7% | 89.7% |

### Example 5: MSCs of Human Patients Suffering from DMD Express Senescence Markers

In order to evaluate whether the senescence of the muscle stem cells in the dystrophic rats reproduces the human pathology of DMD, the regeneration potential and the presence of a senescence signature of the MSCs on human deltoid biopsies from patients suffering from DMD and control patients are evaluated.

DMD muscle biopsies, divided by age classes (1-2, 3-4, 5-6, 7-8 years), and the control biopsies are stained for the embryonic myosin heavy chain (eMHC) in order to mark the myofibers in regeneration (FIG. 5A).

The quantification of the eMHC-positive myofibers showed a progressive reduction in DMD muscle regeneration capacity, with differences labeled between early stage samples (1-2 years) and those of 7-8 years (FIG. 5B).

Co-immunolabeling of Pax7 with Cdkn2a and YH2AX are carried out on histological sections originating from DMD biopsies and control biopsies in order to evaluate the senescence status of human muscle satellite cells.

The muscle sections of patients suffering from DMD had a substantial number of Pax7-positive MSCs which co-expressed Cdkn2a (FIG. 5C-E) and YH2AX (FIG. 5F-H) relative to the control biopsies which expressed barely any Cdkn2a and had low levels of YH2AX (FIG. 5C-H).

In addition, the number of DMD MSCs expressing Cdkn2a increases with age (r=0.84, Pearson correlation, FIG. 7E), without revealing a strong positive correlation between the DMD MSCs expressing YH2AX and the progression of the disease (r=−0.34, Pearson correlation, FIG. 7H).

To conclude, human DMD MSCs express an early and progressive senescent signature, associated with a gradual decline in muscle regeneration potential.

### Example 6: Rat MSCs Express a Signature Specific to EOMs Distinct from that of the TAs

The EOMs are sampled from 12-month-old WT and R-DMDdel52 rats and the cells are isolated and analyzed by scRNAseq with the 10× Genomics platform in order to identify the specific characteristics of the EOMs associated with a regenerative potential effective in DMD.

The same protocol as for the TA samples is used to characterize the EOM samples, identifying an average of 2,505 cells.

The TA and EOM MSC transcriptomic profiles from WT and R-DMDdel52 animals is compared and the specific expression of the Pitx2 gene is detected (Evano, Brendan et al. “Dynamics of Asymmetric and Symmetric Divisions of Muscle Stem Cells In Vivo and on Artificial Niches.” Cell reports vol. 30,10 (2020): 3195-3206.e7. doi:10.1016/j.celrep.2020.01.097; Noden, Drew M, and Philippa Francis-West. “The differentiation and morphogenesis of craniofacial muscles.” Developmental dynamics: an official publication of the American Association of Anatomists vol. 235,5 (2006): 114-218. do:10.1002/dvdy.20697) in the MSCs of the WT and R-DMDdel52 EOMs, confirming the specific identity of these cells (FIG. 6A-C).

As expected, the MSCs isolated from EOMs did not have any senescence signature, Cdkn1a and Cdkn2a being expressed selectively only in the MSCs of the R-DMDdel52 TA (FIG. 6B).

The absence of Cdkn1a and Cdkn2a transcripts is also validated as well as the specific expression of TSHR on muscle satellite cells freshly isolated from EOMs by qRT-PCR (FIG. 6D): The RNA is extracted using the total RNA isolation kit RNAqueous-Micro (Invitrogen) and back-transcribed using SuperScript™ III reverse transcriptase (Invitrogen, 18080093) according to the manufacturer's instructions.

The qPCR is carried out using the StepOnePlus real-time PCR system (Applied Biosystems) and SYBR Green detection tools (Applied Biosystems).

The results are reported as relative gene expression (2-DDCT) using the vehicle-treated cells or the extra-ocular derived myoblasts as a reference. Gene expression is normalized on the expression levels of β-actin.

Co-immunolabeling for Pax7 and the senescence markers is also carried out: Cdkn2a, Cdkn1a and YH2AX (Hernandez-Segura et al. 2018; Wang et al., 2009; Bernadotte et al., 2016; Dungan et al., 2020) (FIG. 6E-G), demonstrating the absence of their expression in the Pax7-positive satellite cells of the EOMs.

Indeed, the percentage quantifications of Pax7±/Cdkn2a±/Cdkn1a±/YH2AX± in the EOMs at 3 weeks, 6 and 12 months demonstrate that there are no differences between the WT and R-DMDdel52 samples.

These data confirm that the MSCs of the EOMs are protected from senescence and retain their myogenic potential in dystrophic rats.

### Example 7: TSHR Signaling Regulates Senescence in the Myoblasts Isolated from the EOMs of R-DMDdel52 Rats

TSHR, the thyroid-stimulating hormone receptor, is specifically expressed in the MSCs of the EOMs (FIG. 6A-C). The expression protein specific for TSHR in the MSCs of the EOMs is validated by immunofluorescence of TSHR on myogenic cells freshly isolated from the extra-ocular muscles and the limbs.

To stain TSHR, the cells were permeabilized for 5 minutes with 0.1% of Triton in 1×PBS, then incubated O/N with the TSHR antibody (CliniSciences, BS-0003R). The following day, the cells were washed in 1×PBS and incubated with Alexa Fluor secondary antibodies for 45 minutes at room temperature. Finally, the nuclei were counterstained with Hoechst (Sigma-Aldrich, B2261). The fluorescence was analyzed with an LSM800 confocal apparatus.

As shown in FIG. 7A, the TSHR is strongly expressed in the myoblasts derived from EOMs in the cell membrane, while the myoblasts isolated from the limb muscles express a low or undetectable level of TSHR.

To evaluate the functional importance of the expression of TSHR in the MSCs of the EOMs, the myoblasts were isolated from the WT EOMs and treated with a selective antagonist of the TSH receptor, ML224 (Neumann et al., 2014).

For this, the myoblasts derived from MSC were treated with 10 μM of ML224 (HY12381-S, CliniSciences) for 2 days (proliferation) or 7 days (differentiation) or 20 μM of forskolin (F3917, Sigma-Aldrich) (following experiment) only for 48 h. The control cells were treated with DMSO (D8418, Sigma Aldrich) as a vehicle for ML224 and forskolin.

The proliferation was measured after 24 h of culture by adding to the culture medium a pulse of 10 mM of 5-ethynyl-20-deoxyuridine (EdU) for 24 h (EdU Click-iT PLUS Kit C10640, Life Technologies).

The inhibition of the signaling of the TSHR has compromised the proliferation of myoblasts with a significant decrease in the Pax7±/MyoD±/EdU± cells relative to the cells treated with the vehicle (FIG. 7B-C).

This experiment is carried out under serum-free conditions in order to prevent any stimulation of TSHR by the presence of TSH in the serum, which explains the lowest proliferative capacity of the myoblasts of the EOMs treated with the vehicle, relative to the data presented above (FIG. 3I).

In addition, the myoblasts of EOMs treated by ML224 exhibited differentiation defects and have compromised the formation of myotubes associated with a significant reduction in the fusion index compared to control cells treated only by the vehicle (FIG. 7D-E).

In addition, the satellite cells of the EOMs in culture treated with the TSHR inhibitor induced the expression of Cdkn1a (p21) and of Cdkn2a (p16) (FIG. 7F), suggesting the acquisition of a senescence signature. It is striking that the myoblasts of the EOMs treated by ML224 have presented a remarkably similar phenotype to that of the R-DMDdel52 limb myoblasts in culture, reminiscent of senescent cells (Moustogiannis, Athanasios et al. “The Effects of Muscle Cell Aging on Myogenesis.” International journal of molecular sciences vol. 22,7 3721. Apr. 2, 2021, doi:10.3390/ijms22073721).

These data demonstrate that TSHR signaling is necessary to prevent the entry into senescence and to maintain the myogenic potential of the MSCs derived from the dystrophic EOMs.

Then, it was hypothesized that the TSHR signaling could also protect the myoblasts derived from DMD MSCs from entering into senescence.

To validate this hypothesis, WT and R-DMDdel52 MSCs isolated from the muscles of the limbs were treated with forskolin, a well-known adenylate cyclase activator (Alasbahi & Melzing, 2012) and TSHR signaling.

It is striking that the R-DMDdel52 myoblasts treated with forskolin exhibited restored proliferation relative to the R-DMDdel52 control cells, reaching the proliferative capacity of the WT cells (FIG. 7G-H).

It is important to note that the R-DMDdel52 myoblasts exposed to forskolin have eliminated the abnormal expression of Cdkn1a (p21) and Cdkn2a (p16), whereas the R-DMDdel52 myoblasts treated with the vehicle have maintained the expression of these senescence markers (FIG. 5I-J).

Finally, the R-DMDdel52 limb myoblasts treated by forskolin exhibited an improved differentiation potential compared to the control R-DMDdel52 cells, forming significantly larger myotubes with a higher fusion index (FIG. 7K-L).

These data suggest that the activation of the AMP/PKA signaling can correct the proliferation and differentiation defects observed in the R-DMDdel52 myoblasts.

### Example 8: Materials and Methods

### Cell Culture

The muscles of the posterior limbs of adult rats were dissected and digested with 3 U/ml of Dispase II (Roche, 4942078001) and 0.5 u/ml of Collagenase A (Roche, 10103586001) for 1 hour in HBSS at 37° C. with slight stirring. The cell suspension was passed through cell filters and centrifuged at 600 g/min for 5 minutes, at 4° C. The supernatant was discarded and the cell pellet was resuspended in 20% fetal bovine serum (Cat #S1810-500 Dominique Dutscher) in DMEM (Cat #41966-029 Gibco) supplemented by 25 ng/mL of bFGF (RP4037, Sigma-Aldrich) with 100 mg/mL penicillin/streptomycin, and placed on 8-chamber slides covered with Matrigel (354234, Life Sciences) (Sarstedt, #94.6140.802). The cells were transduced 24 h after placing and analyzed 72 hours after the transduction.

### Immunofluorescence of the Cultured Cells

The cells were fixed with 4% PFA for 10 minutes at 4° C. and permeabilized with 0.5% Triton in 1×PBS for 6 minutes. After blocking with 10% BSA for 30 min at RT, the cells were incubated O/N at 4° C. with the following primary antibodies: MyoD (Dako, clone 5.8A, M3512), and p16 (Abcam Anti-CDKN2A/p16INK4a, Ab108349). The following day, the cells were washed in 1×PBS and incubated with Alexa Fluor secondary antibodies for 45 min at room temperature. Finally, the nuclei were counter-stained with Hoechst (Sigma-Aldrich, B2261). The fluorescence was analyzed with an LSM800 confocal apparatus. The tomato fluorescence was evaluated with the EVOS M5000 microscope. The lentiviral vector used to overexpress TSHR in our study, pLV [Exp]-Tom-CMV>rTshr [NM_012888.2], was constructed and conditioned by VectorBuilder.

### Isolation of the RNA and RT-qPCR

The RNA was extracted using the total RNA isolation kit RNAqueous-Micro (Invitrogen) and back-transcribed using SuperScript™ III reverse transcriptase (Invitrogen, 18080093) according to the manufacturer's instructions. The qPCR was carried out using the StepOnePlus real-time PCR system (Applied Biosystems) and SYBR Green detection tools (Applied Biosystems). The results are reported as relative gene expression (2-DDCT) using the vehicle-treated cells or the extra-ocular derived myoblasts as a reference. The gene expression of the rat transcript was normalized on the expression levels of the βactin. The sequences of the primers are as follows: Rat Tshr (For CTTTGTCCTGTTCGTCCTGC, Rev AGTGAAGGGACTAGCATTGTC); βactin (For: TGTCACCAACTGGGACGATA, Rev: GGGGTGTTGAAGGTCTCAAA).

### RESULTS

In order to understand whether the overexpression of the TSHR could have an impact on the entry into senescence of the DMD myoblasts, a lentiviral vector carrying the coding sequence of the TSHR and the Tomato reporter gene under the control of the CMV promoter (TSHR Tomato, FIG. 8A) were generated. Wild type myoblasts (WT) and DMD myoblasts were isolated from 6-month-old rats and cultured. The transduction with the coding sequence TSHR Tomato or with a control lentivirus only bearing the Tomato gene was carried out 24 hours after culturing. The cells were analyzed 72 hours after the transduction and the efficiency of the transduction of the WT and DMD myoblasts with TSHR Tomato or a control lentivirus (FIG. 8B) was verified.

The overexpression of TSHR was validated by qPCR, showing a greater expression of the transgene in transduced cells with TSHR Tomato relative to the control cells (FIG. 8C). Interestingly, DMD cells overexpressing TSHR have a lower expression of the p16 senescence marker (FIG. 8D), thus demonstrating that TSHR prevents the entry into senescence of the DMD myoblasts.

In other words, an overexpression of the TSHR receptor would make it possible to prevent and treat DMD.

1-6. (canceled)

7. An activator comprising:
a genetic activator comprising a vector selected from the group consisting of a liposome comprising a thyroid-stimulating hormone receptor (TSHR) protein sequence of SEQ ID NO: 1 or a functional variant having a protein sequence at least 60% identical to the sequence SEQ ID NO: 1, an adenoviral vector comprising the protein sequence of SEQ ID NO: 1 or a functional variant having a protein sequence at least 60% identical to the sequence SEQ ID NO: 1, or a TSHR nucleotide sequence of SEQ ID NO: 2 or a functional variant having a nucleotide sequence at least 60% identical to the sequence SEQ ID NO: 2, an adenoviral vector comprising a CRISPR-Cas9 sequence fused with a transactivator domain preferably selected from VP16, VP64, p65, NCO1A1, FOXO1A to activate the expression of the TSHR, and a combination thereof.

8. The activator according to claim 7, characterized in that said adenoviral vector is an adeno-associated virus (AAV).

9-10. (canceled)

11. An adenoviral vector comprising:
a thyroid-stimulating hormone receptor (TSHR) protein sequence of SEQ ID NO: 1 or a functional variant having a protein sequence at least 60% identical to the sequence SEQ ID NO: 1, or a TSHR nucleotide sequence of SEQ ID NO: 2 or a functional variant having a nucleotide sequence at least 60% identical to the sequence SEQ ID NO: 2 for use thereof as a medicinal product.

12-14. (canceled)

15. The adenoviral vector according to claim 11, wherein said vector is an adeno-associated virus (AAV).

16. The activator of claim 15, wherein the adeno-associated virus is an AAV8 or an AAV9.

17. The activator according to claim 8, wherein the adeno-associated virus is an AAV8 or an AAV9.

18. The activator according to claim 8, further comprising a pharmacological activator.

19. The activator according to claim 18, wherein the pharmacological activator is forskolin.

20. The activator according to claim 19, wherein the genetic activator is the adeno-associated virus (AAV), and the AAV comprises a thyroid-stimulating hormone receptor (TSHR) protein sequence of SEQ ID NO: 1 or a functional variant having a protein sequence at least 60% identical to the sequence SEQ ID NO: 1, or a TSHR nucleotide sequence of SEQ ID NO: 2 or a functional variant having a nucleotide sequence at least 60% identical to the sequence SEQ ID NO: 2.

21. A method of treating muscle stem cells, the method comprising:
providing an activator of a thyroid-stimulating hormone receptor (TSHR) signaling pathway to a human or animal in need thereof,
introducing the activator into a muscle of the human or animal in need thereof, thereby blocking senescence of the muscle stem cells.

22. A method according to claim 21, wherein the activator comprises a genetic activator comprising a vector selected from the group consisting of a liposome comprising a thyroid-stimulating hormone receptor (TSHR) protein sequence of SEQ ID NO: 1 or a functional variant having a protein sequence at least 60% identical to the sequence SEQ ID NO: 1, an adenoviral vector comprising the protein sequence of SEQ ID NO: 1 or a functional variant having a protein sequence at least 60% identical to the sequence SEQ ID NO: 1, or a TSHR nucleotide sequence of SEQ ID NO: 2 or a functional variant having a nucleotide sequence at least 60% identical to the sequence SEQ ID NO: 2, an adenoviral vector comprising a CRISPR-Cas9 sequence fused with a transactivator domain preferably selected from VP16, VP64, p65, NCO1A1, FOXO1A to activate the expression of the TSHR, and a combination thereof.

23. The method according to claim 22, wherein the adenoviral vector of the activator is an adeno-associated virus (AAV).

24. The method according to claim 23, wherein the adeno-associated virus is an AAV8 or an AAV9.

25. The method according to claim 22, wherein the activator further comprises a pharmacological activator.

26. The method according to claim 25, wherein the pharmacological activator is forskolin.

27. The method according to claim 22, wherein the genetic activator is the adeno-associated virus (AAV), and the AAV comprises a thyroid-stimulating hormone receptor (TSHR) protein sequence of SEQ ID NO: 1 or a functional variant having a protein sequence at least 60% identical to the sequence SEQ ID NO: 1, or a TSHR nucleotide sequence of SEQ ID NO: 2 or a functional variant having a nucleotide sequence at least 60% identical to the sequence SEQ ID NO: 2.

28. The method according to claim 21, wherein the human or animal has Duchenne muscular dystrophy or sarcopenia.


SEQUENCE LISTING (8 sequences)

SEQ ID NO: 1 - protein, 764 aa, Homo sapiens
MRPADLLQLVLLLDLPRDLGGMGCSSPPCECHQEEDFRVTCKDIQRIPSLPPSTQTLKLIETHLRTIPSHAFSNLPNISRIYVSIDVTLQQLESHSFYNLSKVTHIEIRNTRNLTYIDPDALKELPLLKFLGIFNTGLKMFPDLTKVYSTDIFFILEITDNPYMTSIPVNAFQGLCNETLTLKLYNNGFTSVQGYAFNGTKLDAVYLNKNKYLTVIDKDAFGGVYSGPSLLDVSQTSVTALPSKGLEHLKELIARNTWTLKKLPLSLSFLHLTRADLSYPSHCCAFKNQKKIRGILESLMCNESSMQSLRQRKSVNALNSPLHQEYEENLGDSIVGYKEKSKFQDTHNNAHYYVFFEEQEDEIIGFGQELKNPQEETLQAFDSHYDYTICGDSEDMVCTPKSDEFNPCEDIMGYKFLRIVVWFVSLLALLGNVFVLLILLTSHYKLNVPRFLMCNLAFADFCMGMYLLLIASVDLYTHSEYYNHAIDWQTGPGCNTAGFFTVFASELSVYTLTVITLERWYAITFAMRLDRKIRLRHACAIMVGGWVCCFLLALLPLVGISSYAKVSICLPMDTETPLALAYIVFVLTLNIVAFVIVCCCYVKIYITVRNPQYNPGDKDTKIAKRMAVLIFTDFICMAPISFYALSAILNKPLITVSNSKILLVLFYPLNSCANPFLYAIFTKAFQRDVFILLSKFGICKRQAQAYRGQRVPPKNSTDIQVQKVTHEMRQGLHNMEDVYELIENSHLTPKKQGQISEEYMQTVL

SEQ ID NO: 2 - RNA, 4308 nt, Homo sapiens
  source 1..4308: mol_type = transcribed RNA
gcagagctgagaatgaggcgatttcggaggatggagaaatagccccgagtcccgtggaaaatgaggccggcggacttgctgcagctggtgctgctgctcgacctgcccagggacctgggcggaatggggtgttcgtctccaccctgcgagtgccatcaggaggaggacttcagagtcacctgcaaggatattcaacgcatccccagcttaccgcccagtacgcagactctgaagcttattgagactcacctgagaactattccaagtcatgcattttctaatctgcccaatatttccagaatctacgtatctatagatgtgactctgcagcagctggaatcacactccttctacaatttgagtaaagtgactcacatagaaattcggaataccaggaacttaacttacatagaccctgatgccctcaaagagctccccctcctaaagttccttggcattttcaacactggacttaaaatgttccctgacctgaccaaagtttattccactgatatattctttatacttgaaattacagacaacccttacatgacgtcaatccctgtgaatgcttttcagggactatgcaatgaaaccttgacactgaagctgtacaacaatggctttacttcagtccaaggatatgctttcaatgggacaaagctggatgctgtttacctaaacaagaataaatacctgacagttattgacaaagatgcatttggaggagtatacagtggaccaagcttgctggacgtgtctcaaaccagtgtcactgcccttccatccaaaggcctggagcacctgaaggaactgatagcaagaaacacctggactcttaagaaacttccactttccttgagtttccttcacctcacacgggctgacctttcttacccaagccactgctgtgcttttaagaatcagaagaaaatcagaggaatccttgagtccttgatgtgtaatgagagcagtatgcagagcttgcgccagagaaaatctgtgaatgccttgaatagccccctccaccaggaatatgaagagaatctgggtgacagcattgttgggtacaaggaaaagtccaagttccaggatactcataacaacgctcattattacgtcttctttgaagaacaagaggatgagatcattggttttggccaggagctcaaaaacccccaggaagagactctacaagcttttgacagccattatgactacaccatatgtggggacagtgaagacatggtgtgtacccccaagtccgatgagttcaacccgtgtgaagacataatgggctacaagttcctgagaattgtggtgtggttcgttagtctgctggctctcctgggcaatgtctttgtcctgcttattctcctcaccagccactacaaactgaacgtcccccgctttctcatgtgcaacctggcctttgcggatttctgcatggggatgtacctgctcctcatcgcctctgtagacctctacactcactctgagtactacaaccatgccatcgactggcagacaggccctgggtgcaacacggctggtttcttcactgtctttgcaagcgagttatcggtgtatacgctgacggtcatcaccctggagcgctggtatgccatcaccttcgccatgcgcctggaccggaagatccgcctcaggcacgcatgtgccatcatggttgggggctgggtttgctgcttccttctcgccctgcttcctttggtgggaataagtagctatgccaaagtcagtatctgcctgcccatggacaccgagacccctcttgctctggcatatattgtttttgttctgacgctcaacatagttgccttcgtcatcgtctgctgctgttatgtgaagatctacatcacagtccgaaatccgcagtacaacccaggggacaaagataccaaaattgccaagaggatggctgtgttgatcttcaccgacttcatatgcatggccccaatctcattctatgctctgtcagcaattctgaacaagcctctcatcactgttagcaactccaaaatcttgctggtactcttctatccacttaactcctgtgccaatccattcctctatgctattttcaccaaggccttccagagggatgtgttcatcctactcagcaagtttggcatctgtaaacgccaggctcaggcataccgggggcagagggttcctccaaagaacagcactgatattcaggttcaaaaggttacccacgagatgaggcagggtctccacaacatggaagatgtctatgaactgattgaaaactcccatctaaccccaaagaagcaaggccaaatctcagaagagtatatgcaaacggttttgtaagttaacactacactactcacaatggtaggggaacttacaaaataatagtttcttgaatatgcattccaatcccatgacacccccaacacatagctgccctcactcttgtgcaggcgatgtttcaatgtttcatggggcaagagtttatctctggagagtgattagtattaacctaatcattgcccccaagaaggaagttaggctaccagcatatttgaatgccaggtgaaatcaaaataatctacactatctagaagactttcttgatgccaagtccagagatgtcattgtgtaggatgttcagtaaatattaactgagctatgtcaatatagagcttctcagttttgtataacatttcatactaaagattcagcaaatggaaaatgctattaatttggttggtgaccacaagataaaatcagtcccacgttggctcagttcaactagatgttccctgatacaaagagaacttgatttccttaaaactgaaaagccaaacacagctagctgtcatacaagaaacagctattatgagacatgaaggagggtaagaattagctttaagttttgttttgctttgttttgttttttaactcaacctattaatcatctcttcacaagaatccacctgatgtgaccaagctattatgtgttgcctggaaaaactggcaagatttcagcttatgtggcctagcaaactaagaattgctcttcttggccagcctcatagcataaaagatgtgaactctaggaagtctttctgagtagcaataagtgggaattatgggcagagcacactcaatcccctgttgattaataaaacaggctggacactaattaactatgggacttaaatctgtagaaatgaaggagtccaatagcttcttccaattttaaaactctagtacatccctttccctcaaatatatatttctaagataaagagaaagaagagcactaagtaagtagaatctgtttttcctattttgtagggctgctgactcctagtccttgaagcctagacacatgacccaggaaatttttcctttgtttcacttttgattatgatgtctgagccaaaaattcaattaagtaaacatactcgcctggatctgaatcattcatttaattactagatctacccagctgttatatcaggccaaaaacagattcgtgtttatataaaagagtaaacgatggttgcaaattttggctatttagagttgctacttcactatgaagagtcacttcaaaacacttcgcttgtctttagggatgatttttgccatttccagtccacggtatgatactaaagctgtcaagagaggtttcttcttttctgaaactgccagctctttccagccctgttgatcactggacataaagcttcttttccccaataattcttctttacttaaaatagtcaggatctttatctacagatgtactctccaggttacctgtgatgatagccccctaatgtcctgctagaaaagtctccaagcagagatgacattacttctgaatgctcataaaccacaccatgaaataaaagctctttgttgttttaagattgtgaagtgtcgttaatgggtccccacagatggtccctgctggactcacctggaatctctccacagccatacccactcatcactatcattgagacctgcacatcttaatagaaatattataaacatcgaaaatcatgacttacctagaagttcgcttgtaactaatgaaattaaacaaatgtgttgccttttgtcatgtgtttctctcctgtgacatttcaaaatatcacatcttgataaataatgtgtttcatcttgaatagctgaactaattgctttggaaacagagtcctagaaaagtgacttcaacagaattgttactaaaatttgcactcacaacatgaaataaattttcttcctatggaataatcgtgccaa

SEQ ID NO: 3 - DNA, 25 bp, synthetic construct
  source 1..25: mol_type = other DNA
ctaacgcatttaaaatatgctgtca

SEQ ID NO: 4 - DNA, 25 bp, synthetic construct
  source 1..25: mol_type = other DNA
gttggcttagctcaacaaccaagat

SEQ ID NO: 5 - DNA, 20 bp, synthetic construct
  source 1..20: mol_type = other DNA
ctttgtcctgttcgtcctgc

SEQ ID NO: 6 - DNA, 21 bp, synthetic construct
  source 1..21: mol_type = other DNA
agtgaagggactagcattgtc

SEQ ID NO: 7 - DNA, 20 bp, synthetic construct
  source 1..20: mol_type = other DNA
tgtcaccaactgggacgata

SEQ ID NO: 8 - DNA, 20 bp, synthetic construct
  source 1..20: mol_type = other DNA
ggggtgttgaaggtctcaaa